(19) United States
(12) Patent Application Publication
(10) Pub. No.: US 2013/0172921 A1
(43) Pub. Date: Jul. 4, 2013

(54) METHOD FOR TREATMENT OF EMPHYSEMA

(71) Applicant: TERUMO KABUSHIKI KAISHA, Shibuya-ku (JP)

(72) Inventors: Yasushi KINOSHITA, Fujinomiya-shi (JP); Wataru KARINO, Ashigarakami-gun (JP); Yuichi TADA, Ashigarakami-gun (JP); Taiga NAKANO, Ashigarakami-gun (JP); Tomonori HATTA, Ashigarakami-gun (JP)

(73) Assignee: TERUMO KABUSHIKI KAISHA, Shibuya-ku (JP)

(21) Appl. No.: 13/727,248

(22) Filed: Dec. 26, 2012

Related U.S. Application Data
(60) Provisional application No. 61/580,735, filed on Dec. 28, 2011.

(51) Int. Cl. A61B 17/34

(52) U.S. Cl. CPC A61B 17/34
USPC 606/185

(57) ABSTRACT

The present invention provides a method for treatment of emphysema, including: (a) a step of inserting and placing first through-hole forming means in emphysematous lung area, forming a first through-hole in the emphysematous lung area by puncture, and, after contact of the first through-hole forming means with pleura, detaching the pleura from the lung area to form a detachment space; (b) a step of inserting and placing second through-hole forming means in normal lung area, and forming a second through-hole in the lung area by puncture; and (c) a step of causing the first through-hole and the second through-hole to communicate with each other through the detachment space formed in the step (a).

The patent or application file contains at least one drawing executed in color. Copies of this patent with color drawing(s) will be provided by the Office upon request and payment of the necessary fee.

### BACKGROUND OF THE INVENTION

1. Field of the Invention

The present invention relates to a method for treatment of emphysema.

2. Description of the Related Art

Chronic obstructive pulmonary disease (COPD) means a diverse group of pulmonary diseases in which normal respiration is hampered due to inflammation caused on the airway or alveoli by smoking or dust, in other words, diseases in which the lung or lungs are obstructed by the presence of at least one disease selected from among bronchial asthma, emphysema and chronic bronchitis. The two most serious conditions related to COPD are chronic bronchitis and emphysema.

Of these diseases, the emphysema is a disease in which elasticity of alveolar walls is lost due to inflammation, whereby elasticity and contractility of alveoli themselves are lowered, and cell walls are broken to cause accumulation of air, resulting in chronically lowered pulmonary functions. In addition, bronchioles connected to the emphysematous alveoli are also thickened by inflammation, and are obstructed due to a rise in the intrathoracic pressure at the time of expiration. A combination of these main factors leads to difficulty in discharge of air from the emphysematous alveoli, although inspiration is performed without trouble. By the same token, the lung in which air has accumulated intumesces to compress normal alveoli, as well, thereby lowering the ventilation efficiency. More specifically, while normal lung parenchyma shrinks at the time of expiration, emphysematous lung parenchyma would not return to its contracted state once expanded by respiration. Therefore, the emphysematous lung parenchyma cannot perform sufficient expiration. Moreover, the alveolar effective area and vascular bed (capillary vessels extending in all directions throughout the surfaces of alveoli) are decreased, so that the ventilation capability of the lung as a whole is lowered. In addition, since elastin and collagen and the like are broken by inflammation, the lung is lowered in elasticity, and becomes unable to keep pulling and spreading the airway, so that the bronchial tube is in an easily deformable state. Accordingly, when the lung shrinks at the time of expiration, the bronchial tube is narrowed by being compressed by the surrounding alveoli filled with air, resulting in hyperinflation of the lung and difficult discharge of air.

Examples of the general therapeutic method for COPD include a concentrated oxygen inhalation method, medicinal treatments, respiratory rehabilitations, a noninvasive positive pressure respiration method using a nasal mask, lung volume reduction surgery, and transbronchial treatment methods intended to achieve minimal invasive lung volume reduction. The respiratory rehabilitations, such as pursed-lip breathing and abdominal breathing, and the concentrated oxygen inhalation method are often employed in situations where sufficient absorption of oxygen from air is impossible because of severely impaired pulmonary functions. These methods, however, can only mitigate the symptom, and cannot be said to be effective therapeutic methods. Examples of the medicinal treatments include: a method in which a bronchodilator is used to help patency of the airway in the lung and to reduce shortness of breath; a method in which a steroid for inhalation administration or oral administration is used to reduce inflammation in the airway; a method in which an antibiotic is employed to prevent and treat additional infection; and a method in which an expectorant is used to remove mucus from the airway. Although every one of these medicinal treatments helps control the emphysema and mitigate the symptom, it cannot be said to be an effective therapeutic method. In addition to the above, there are surgical treatment methods such as the lung volume reduction surgery in which a damaged part of an excessively expanded lung is removed to promote expansion of normal lung parts; however, this surgery imposes a considerable burden on the patient. As another surgical treatment method, there is lung transplantation, but this involves difficulties in securing substituent lungs. Furthermore, the noninvasive positive pressure respiration method using a nasal mask has a problem that the nasal mask would cause pressure necrosis or pressure sore of the surroundings of the nose (especially, the root of the nose). Besides, the transbronchial treatment methods intended to achieve lowly invasive lung volume reduction include: a method in which a one-way valve for restricting air inflow is placed at a bronchial part suffering chronic obstruction; a bypass technique applied to the bronchial wall; and an emphysema peripheral part reduction surgery in which placement of a shape memory alloy wire and deformation thereof are utilized.

As the method in which a one-way valve for limiting the air inflow is placed indwelling, there is known, for example, the technology disclosed in U.S. Pat. No. 6,258,100. In this system, a structure having a mechanism of one-way valve is placed indwelling in the bronchial tube, so as to hamper inspiration from flowing in toward the terminal of the lung.

A technology of bypass formation for the bronchial wall is disclosed in Japanese Patent Laid-Open No. 2004-358241. In this technology, the trachea of a patient suffering chronic obstructive pulmonary disease and a predetermined part of the patient's lung are connected by a conduit, and the trachea and an internal volume part of the lung are put into fluid communication with each other, whereby air trapped in the lung is discharged to the outside of the patient's body.

An emphysema peripheral part reduction surgery in which placement of a shape memory alloy wire and deformation thereof are utilized is disclosed in JP-T-009-529966. In this technology, a filamentous implant made of a shape memory alloy is placed in an emphysematous part, thereafter the implant is rapidly cooled to change the implant into its bent configuration, thereby entangling the emphysematous part and reducing the lung volume.

### SUMMARY OF THE INVENTION

However, the lung volume reduction valve described in the U.S. Pat. No. 6,258,100 has the problem that where such a structure is placed indwelling, it becomes difficult to make access to the distal side of the structure (Mark L. Mathis, PneumRx, Inc., U.S. Pat. No. 7,549,984). In addition, the bypass forming surgery applied to the bronchial wall described in Japanese Patent Laid-Open No. 2004-358241 has the problem that the burden on the patient is still heavy, and, once the bypass forming surgery is applied to the bronchial wall, it is difficult to restore the body into the state before the bypass forming surgery. Further, the emphysema peripheral part reduction surgery described in JP-T-2009-529966 has the risk of entangling normal alveoli; moreover, there is also the problem that it is difficult to restore the body into the state before the surgery, like in the case of the Japanese Patent Laid-Open No. 2004-358241.

Therefore, as above-mentioned, no effective therapeutic method for emphysema is present in the relevant field at present. Besides, every one of the therapeutic methods using the inventions disclosed in the related art documents mentioned above is so to speak a method in which the emphysematous part is removed.

In view of the foregoing, according to the present invention, there is provided a technique by which it is possible, by using a part of a living body organ as a bypass, to form a bypass in such a manner that the state before the application of the technique can be recovered even after the application of the technique, or in such a manner as not to change the configuration of the lung parenchyma inclusive of the lesion part, and it is possible to reduce the risk of infection or inflammation. Also provided is a device for use in the treatment according to the technique.

Thus, in order to solve the above-mentioned problems, it is an object of the present invention to provide a technique in which emphysematous lung parenchyma or emphysematous airway and normal lung parenchyma or normal airway are connected to each other by utilizing pleura, as a therapeutic method intended to achieve treatment of only a target emphysematous part, and a device for use in the treatment based on the technique.

According to the present invention, there is a method for treatment of emphysema, including:

a step (a) of inserting and placing first through-hole forming means in emphysematous lung area, forming a first through-hole in the emphysematous lung area by puncture, and, after contact of the first through-hole forming means with pleura, detaching the pleura from the lung area to form a detachment space;

a step (b) of inserting and placing second through-hole forming means in normal lung area, and forming a second through-hole in the lung area by puncture; and

a step (c) of causing the first through-hole and the second through-hole to communicate with each other through the detachment space formed in the step (a).

According to the present invention, there is also a method for treatment of emphysema, including:

a step (k) of inserting and placing first through-hole forming means in emphysematous lung area, forming a first through-hole in the lung area by puncture, and, after contact of the first through-hole forming means with pleura, detaching the pleura from the lung area to form a detachment space; and

a step (l) of inserting and placing the first through-hole forming means in normal lung are by way of the detachment space formed in the step (k), and forming a second through-hole in the lung area by puncture.

In addition, the device for use in the treatment according to the present invention is as shown in the following paragraphs (1) to (8).

(1) A pleural bypass forming system including:

a lung parenchyma or airway penetrating element including a long body wire capable of being inserted and passed in a respiratory region, and a flexible section which is provided at the distal end of the body wire, penetrates lung parenchyma or airway to form a through-hole, and, upon making contact with pleura, detaches the pleura from the lung parenchyma or the airway;

a through-hole broadening element provided at its distal end with a through-hole broadening member for broadening the through-hole; and

a pleura detaching element including an expandable and contractible elastic member which is passed through the broadened through-hole to be inserted into a detachment space formed by detaching the pleura from the lung parenchyma or the airway and expands the detachment space, and an operating member for operating the elastic member from the proximal end.

(2) The pleural bypass forming system as described in the paragraph (1) above, further including a pleural bypass forming element which includes: a guide wire inserted through the broadened through-hole into the detachment space; a catheter provided with at least one lumen in which the guide wire can be inserted and passed; a tubular indwelling body contained on the distal end in the catheter; and placement means for placing the tubular indwelling body in the through-hole.

(3) The pleural bypass forming system as described in the paragraph (2) above, further including placement means for placing the tubular indwelling body in the detachment space.

(4) The pleural bypass forming system as described in the paragraph (2) above, wherein the tubular indwelling body establishes communication between lung parenchyma parts or between airway parts.

(5) The pleural bypass forming system as described in any one of the paragraphs (1) to (3) above, wherein the flexible section is provided at the distal end of the body wire, penetrates the lung parenchyma or airway, and, upon making contact with pleura, deforms along the pleura.

(6) The pleural bypass forming system as described in any one of the paragraphs (1) to (5) above, wherein the through-hole broadening member is tapered off at its distal end and is in the shape of a tube having at least one lumen in which the lung parenchyma or airway penetrating element can be inserted and passed.

(7) The pleural bypass forming system as described in the paragraph (2) above, wherein the placement means is composed of a pusher by which the tubular indwelling body contained on the distal end in the catheter is protruded by an operation on the proximal end of the catheter.

(8) A pleural bypass forming system including:

a lung parenchyma or airway penetrating element including a long body wire capable of being inserted and passed into a respiratory region, and a flexible section which is provided at the distal end of the body wire, penetrates the lung parenchyma or airway to form a through-hole, and, upon making contact with the pleura, detaches the pleura from the lung parenchyma or airway;

a pleura detaching element including an expandable and contractible elastic member which is passed through the through-hole to be inserted into a detachment space formed by detaching the pleura from the lung parenchyma or airway and expands the detachment space, and an operating member for operating the elastic member from the proximal end; and a puncture positioning section including a catheter in which the lung parenchyma or airway penetrating element can be inserted and passed, and a suction section which is provided at the distal end of the catheter and by which the lung parenchyma or the airway is fixed by suction.

The above and other objects, features and advantages of the present invention will become clear from the following description of the preferred embodiments.

### BRIEF DESCRIPTION OF THE DRAWINGS

FIG. 1 is a schematic view relating to step (a) according to the present invention;

FIG. 2 is a schematic view relating to the step (a) according to the invention;

FIG. 3 is a schematic view relating to the step (a) according to the invention;

FIG. 4 is a schematic view relating to a preferred example of first through-hole forming means for use in the step (a) according to the invention;

FIG. 5 is a schematic view relating to the step (a) according to the invention;

FIG. 6 is a schematic view relating to step (b) according to the invention;

FIG. 7 is a schematic view relating to step (c) according to the invention;

FIG. 8 is a schematic view relating to step (d) according to the invention;

FIG. 9 is a schematic view relating to the step (d) according to the invention;

FIG. 10 is a schematic view relating to the step (d) according to the invention;

FIG. 11 is a schematic view relating to step (e) according to the invention;

FIG. 12 is a schematic view relating to step (f) according to the invention;

FIGS. 13A to 13C show schematic views relating to a preferred example of a pleura detaching element for use in the step (f) according to the invention;

FIG. 14 is a schematic view relating to step (g) and step (h) according to the invention;

FIG. 15 is a schematic view relating to a preferred example of a pleural bypass forming element for use in the step (g) according to the invention;

FIGS. 16A and 16B are schematic views relating to a preferred example of placement means in the pleural bypass forming element for use in the step (g) according to the invention;

FIG. 17 is a schematic view relating to a preferred example of a pleural bypass forming system for use in the present invention;

FIG. 18 is a schematic view relating to a first embodiment according to the invention;

FIG. 19 is a schematic view relating to a second embodiment according to the invention;

FIG. 20 is a schematic view relating to a third embodiment and step (i) according to the invention;

FIG. 21 is a schematic view relating to the third embodiment and step (j) according to the invention;

FIG. 22 is a schematic view relating to the third embodiment and step (k) according to the invention;

FIG. 23 is a schematic view relating to the third embodiment and the step (k) according to the invention; and

FIG. 24 is a schematic view relating to the third embodiment and the step (i) according to the invention.

### DETAILED DESCRIPTION OF THE PREFERRED EMBODIMENTS

A first point of the present invention lies in a method for treatment of emphysema, including:
(a) a step of inserting and placing first through-hole forming means in emphysematous lung area, forming a first through-hole in the emphysematous lung area by puncture, and, after contact of the first through-hole forming means with pleura, detaching the pleura from the lung area to form a detachment space;
(b) a step of inserting and placing second through-hole forming means in normal lung area, and forming a second through-hole in the lung area by puncture; and
(c) a step of causing the first through-hole and the second through-hole to communicate with each other through the detachment space formed in the step (a).

According to the technique of the present invention, the treatment is conducted by use of a catheter, without any surgical operation, so that burden on the patient is very slight (namely, the technique is lowly invasive). Further, normal alveoli and emphysematous alveoli can be made to communicate with each other through a detachment space formed between the lung and the pleura near the lung surface. As a result, air accumulating in the emphysematous part is exhaled and discharged through the detachment space, which is a subpleural air accumulation area, and a normal alveolar part.

According to the present invention, the normal alveoli and the emphysematous alveoli can be made to communicate with each other by utilizing the detachment space formed between the lung and the pleura near the lung surface. Consequently, the air accumulating in the emphysematous part is exhaled and discharged through the detachment space, which is a subpleural air accumulation area, and the normal alveolar part.

Incidentally, the term “treatment” used herein means medical activities aiming to cure, mitigate, alleviate, repair, prevent, or ameliorate emphysema, an emphysematous symptom, or a diseased condition following the emphysema.

In addition, the term “airway” used herein is a name for a body part including trachea, principal bronchus, lobar bronchus, bronchial tube, bronchioles, and terminal bronchioles, whereas the term “lung parenchyma” is a name for a body part including respiratory bronchioles, alveolar ducts, and alveolar sacs.

The method for treatment of emphysema according to the present invention may optionally include steps (d) to (j) described below, in addition to the above-mentioned steps (a) to (c).

Besides, the step (k) and step (l) may be carried out, in place of the steps (a), (b) and (c). Specifically, a second point of the present invention resides in a method for treatment of emphysema, including:
(k) a step of inserting and placing first through-hole forming means in emphysematous lung area, forming a first through-hole in the lung area by puncture, and, after contact of the first through-hole forming means with pleura, detaching the pleura from the lung area to form a detachment space; and
(l) a step of inserting and placing the first through-hole forming means in normal lung are by way of the detachment space formed in the step (k), and forming a second through-hole in the lung area by puncture.

In the case where the step (k) and step (l) are conducted as the method for treatment of emphysema according to the present invention, also, the steps (d) to (j) described below may optionally be carried out in addition to the step (k) and step (l).

Now, preferred embodiments of the present invention, in terms of the steps, will be described in detail below referring to the drawings.

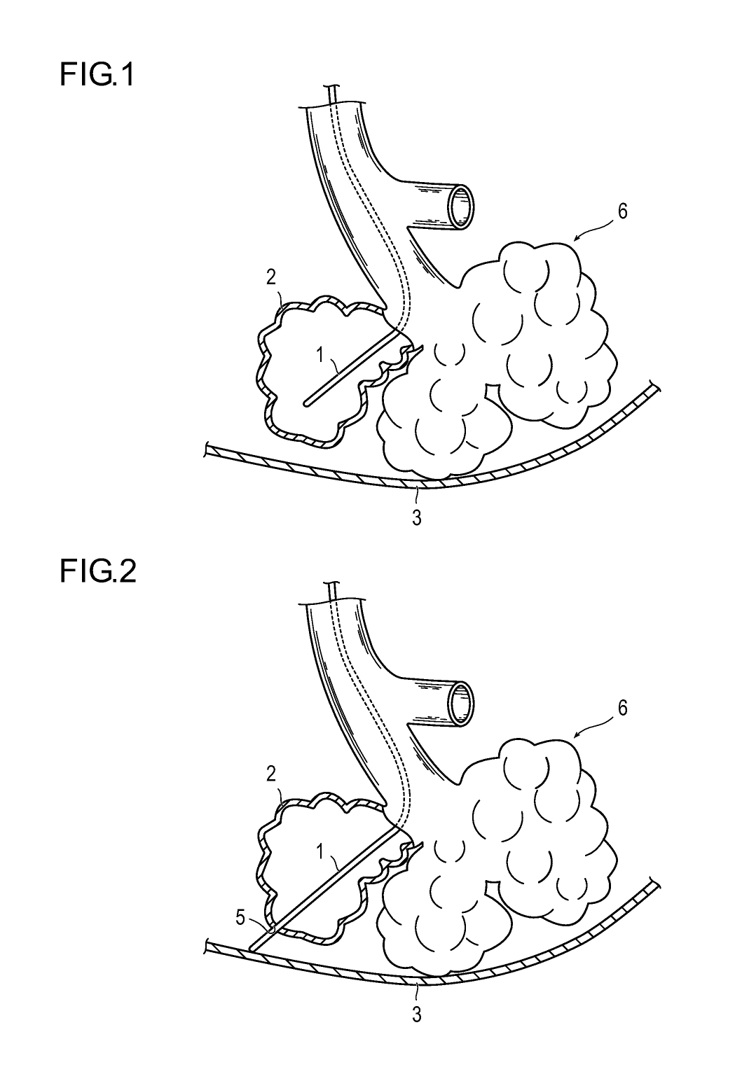

FIGS. 1 to 21 (exclusive of FIGS. 4, 9, 13, 15, 16 and 17) are schematic sectional views showing the sequence of steps in the method according to the present invention. While the steps will be described in detail below referring to FIGS. 1 to 21, the steps are not restricted to the following embodiments.

“Step (a)”

In this step, as shown in FIG. 1, first through-hole forming means 1 is inserted and placed in emphysematous lung parenchyma 6 or emphysematous airway (not shown), a first through-hole 5 is formed in the lung parenchyma or airway by puncture, and, upon contact of the first through-hole forming means 1 with a pleura 3, the pleura is detached from the alveoli 2 to form a detachment space 4. Specifically, the first through-hole forming means 1 is inserted through the patient's oral cavity or nasal cavity or the like into bronchial tube or bronchioles communicating with a respiratory region contained in emphysematous alveoli or alveolar sacs (hereinafter referred also to simply as “emphysematous lung parenchyma”) 6 or emphysematous airway. After it is checked, under radioscopy if needed, whether or not a distal section of the first through-hole forming means 1 has been delivered to a desired site, the first through-hole forming means 1 is pushed out toward the distal side. As a result, the emphysematous lung parenchyma 6 or emphysematous airway is punctured, whereby the first through-hole 5 is formed to penetrate only the emphysematous part without breaking the pleura 3 (see FIG. 2).

The pleura covering the surface of the lung as a whole and the surface wall of the lung parenchyma or airway (inclusive of bronchioles) are different in mechanical strength such as perforation strength or bursting strength; specifically, the pleura is higher in mechanical strength and is less susceptible to breaking. Therefore, it is possible for the first through-hole forming means 1 to penetrate only the emphysematous part without breaking the pleura.

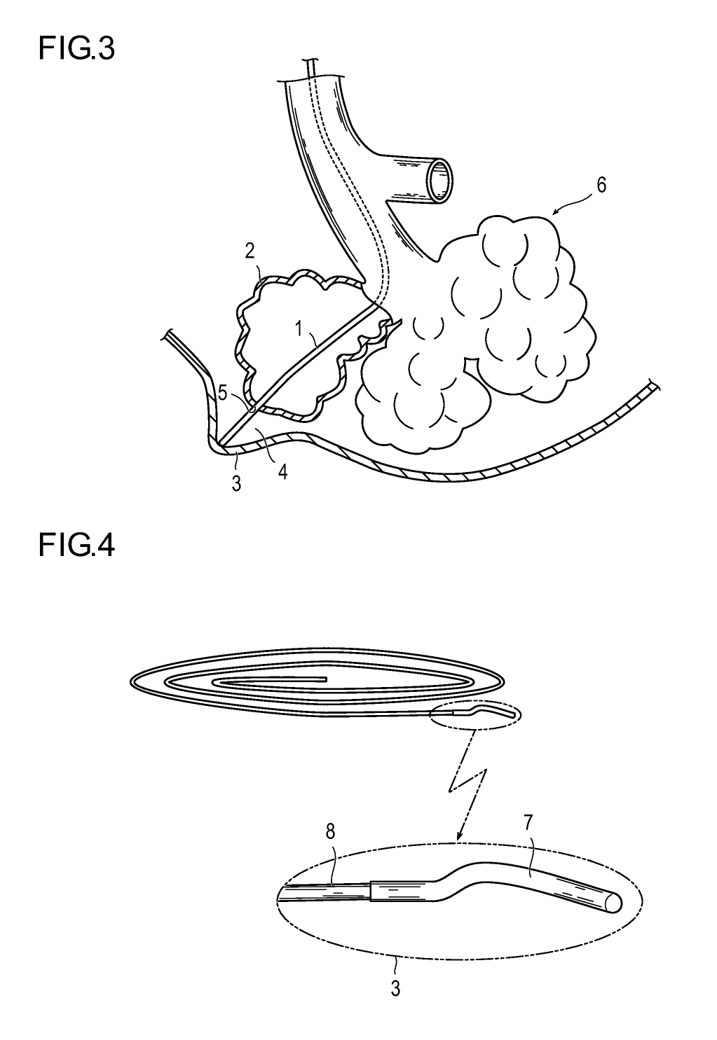

Besides, when the first through-hole forming means 1 in the present invention is pushed out toward the distal side by an operation on the proximal end, a distal section of the first through-hole forming means 1 is brought into contact with the pleura 3 by enhancing the pushing-out force, and, while checking the situation under radioscopy if necessary, the pleura 3 is detached from the alveoli 2 to form the detachment space 4 (see FIG. 3).

As a result, the emphysematous lung parenchyma 6 or emphysematous airway and the detachment space are made to communicate with each other, whereby it is made possible for air in the emphysematous lung parenchyma 6 or emphysematous airway to flow into the detachment space 4. In addition, the size of the detachment space formed in the step (a) is appropriately controlled according to the doctor's judgment.

The first through-hole forming means is preferably composed of a filamentous airway penetrating element including a long body wire capable of being inserted and passed into the respiratory region, and a flexible section which is provided at the distal end of the body wire and penetrates the lung parenchyma or airway to form the through-hole and, upon making contact with the pleura, detaches the pleura from the alveoli. For instance, as represented by a preferred example of the first through-hole forming means in FIG. 4, a filamentous body having a flexible section 7 attached to the distal end of the long body wire 8 is employed.

The filamentous body is used while utilizing the difference in mechanical strength between the alveoli and the pleura. Since the filamentous body in the present invention is provided with the flexible section at the distal end thereof, the filamentous body can penetrate the lung parenchyma or airway without breaking the pleura covering the lung surface. In addition, when the distal end of the filamentous body 1 in the present invention comes into contact with the pleura 3 after penetrating the lung parenchyma or airway (see FIG. 2), the distal end can detach the pleura 3 from the alveoli 2 without breaking the pleura 3 (see FIG. 3).

The thickness of the flexible section in the present invention is preferably 0.05 to 3.0 mm, more preferably 0.3 to 2.0 mm, further preferably 0.4 to 0.9 mm.

From the necessity for reaching the peripheral side of the bronchioles, the thickness of the flexible section is desirably not more than 2 mm. Besides, the risk of puncturing the pleura is enhanced where the sectional area at the most distal end of the flexible section is too small. Therefore, the thickness of the flexible section is preferably in the range of 0.4 to 0.9 mm.

The length of the flexible section in the present invention is preferably 1 to 100 mm, more preferably 5 to 50 mm, and further preferably 10 to 30 mm, from the viewpoint that the length of the flexible section after penetrating the alveolus is easy to confirm under radioscopy and that the flexible section would not thereafter penetrate other alveoli.

Preferred examples of the material forming the flexible section in the present invention include stainless steels such as SUS316L, pure metals such as Ta, superelastic alloys such as Ni—Ti superelastic alloys, and thermoplastic resins such as urethane resins and nylons. These materials can be used either singly or as a composite material.

In addition, the first through-hole forming means in the present invention preferably has a radiopaque property. Therefore, in the case of selecting a metallic material as the material for the flexible section, it is preferable to select a metallic material from among stainless steels, superelastic alloys, cobalt alloys, noble metals such as gold, platinum, tungsten, etc. and their alloys. Especially, where the flexible section of the first through-hole forming means is formed from a radiopaque material such as noble metals, the first through-hole forming means is provided with radiopacity, and can be inserted into a living body while checking the position of its distal end under fluoroscopy, in a favorable manner.

In addition, where a thermoplastic resin is selected as the material for the flexible section, a radiopaque material containing a substance selected from among iodine, barium, bismuth, boron, bromine, calcium, gold, platinum, silver, iron, manganese, nickel, gadolinium, dysprosium, tungsten, tantalum, stainless steel, Nitinol, and their compounds such as barium sulfate, and their solutions/dispersions (e.g., physiological saline); amidotrizoic acid (3,5-diacetamino-2,4,6-triiodobenzoic acid), sodium meglumine amidotrizoate, meglumine amidotrizoate, sodium iotalamate, meglumine iotalamate, meglumine iotroxate, iotrolan, ioxaglic acid, ioxilan, Iopamidol, iopromide, iohexyl, ioversol, iomeprol; iodized fatty acid ethylester of poppy oil (for example, Lipiodol™, which is poppy seed oil wherein carbon atom is iodized), etc., may be added to the flexible section material in a particulate state or as it is, and either singly or in the form of a mixture of two or more of them. Alternatively, the radiopaque material may be provided in a layer form, for example, on the surface of the flexible section by coating or the like.

The shape in section orthogonal to the axis of the flexible section in the present invention is not particularly restricted, and may be appropriately selected from among circular shape, elliptic shapes, polygonal shapes, etc.

The body wire in the present invention is not specifically restricted, insofar as it is a long member. For instance, the thickness of the body wire is preferably 0.1 to 3.0 mm, more preferably 0.3 to 2.0 mm. The length of the body wire is preferably 200 to 3,000 mm, more preferably 600 to 1,200 mm.

Examples of the material for the body wire in the present invention include polymer materials, metallic materials, carbon fiber, and ceramics. These materials may be used either singly or in appropriate combination. The material is not specifically restricted so long as it has certain degrees of rigidity and elasticity. It is preferable, however, that the material is a biocompatible material. Preferred examples of the material include metals, polymer materials and carbon fiber, and more preferable examples include metals and polymer materials.

Specifically, preferred examples of the polymer materials include polyolefins such as polyethylene, polypropylene, etc., aromatic polyesters such as polyethylene terephthalate, etc., fatty polyesters such as polylactic acid, polyglycolic acid, etc., cellulose polymers such as cellulose acetate, cellulose nitrate, etc., and fluorine-containing polymers such as polytetrafluoroethylene, tetrafluoroethylene-ethylene copolymer, etc.

Preferable examples of the metallic materials include stainless steels, tantalum, tantalum alloys, titanium, titanium alloys, nickel-titanium alloys (superelastic alloys), tantalum-titanium alloys, nickel-aluminum alloys, Inconel, gold, platinum, platinum-iridium alloys, tungsten, tungsten alloys, and cobalt alloys. Among stainless steels, preferred is SUS316L, which is the best in corrosion resistance. Among the cobalt alloys, preferred are MP35N, L605 and the like. Among tungsten alloys, preferred are W-Rh25% and W-Rh26%.

In addition, the body wire in the present invention is preferably coated with a known low-friction material such as polytetrafluoroethylene, or a known biocompatible material such as polylactic acid, phospholipids polymers (see, for example, Japanese Patent Laid-Open No. 2005-239988), and PEG, on the surface thereof. Covering the surface of the body wire with the low-friction material or the biocompatible material can enhance the operability or the biocompatibility.

Incidentally, the body wire in the present invention may also have radiopacity, like the above-mentioned flexible section. In that case, the body wire can be provided with the X-ray-opaque material or radiopaque material, by the same technique as in the case of the flexible section.

The first through-hole forming means in the present invention is preferably a filamentous body having a configuration wherein a flexible section capable of penetrating the lung parenchyma or airway but incapable of breaking the pleura is connected to the distal end of the above-mentioned long body wire, as shown in FIGS. 1 to 4.

A preferable example of the first through-hole forming means in the present invention is a configuration wherein the flexible section is attached to the body wire. In this case, the attaching method is not specifically restricted. For example, in the case where the flexible section is formed from an organic material and the body wire is formed from a metallic material, the flexible section is preferably attached to the body wire by welding or crimping. Where both the flexible section and the body wire are formed from metallic materials, they are preferably attached to each other by joining.

In addition, it is preferable for the flexible section in the present invention to be provided at the distal end of the body wire, to penetrate lung parenchyma or airway, and to deform along the pleura upon making contact with the pleura.

Specifically, the first through-hole forming means 1 in the present invention is protruded along the axial direction toward the distal side by an operation on the proximal end. As a result, the flexible section 9 attached to the distal end of the body wire penetrates the lung parenchyma or airway, and, upon making contact with the pleura 3 covering the lung surface, the flexible section deforms along the pleura 3. Therefore, the flexible section 9 can penetrate the lung parenchyma or airway without breaking the pleura 3 covering the lung surface (see FIG. 5).

Besides, the filamentous body as a preferable embodiment of the first through-hole forming means in the present invention may be used as a guide wire in the present invention.

“Step (b)”

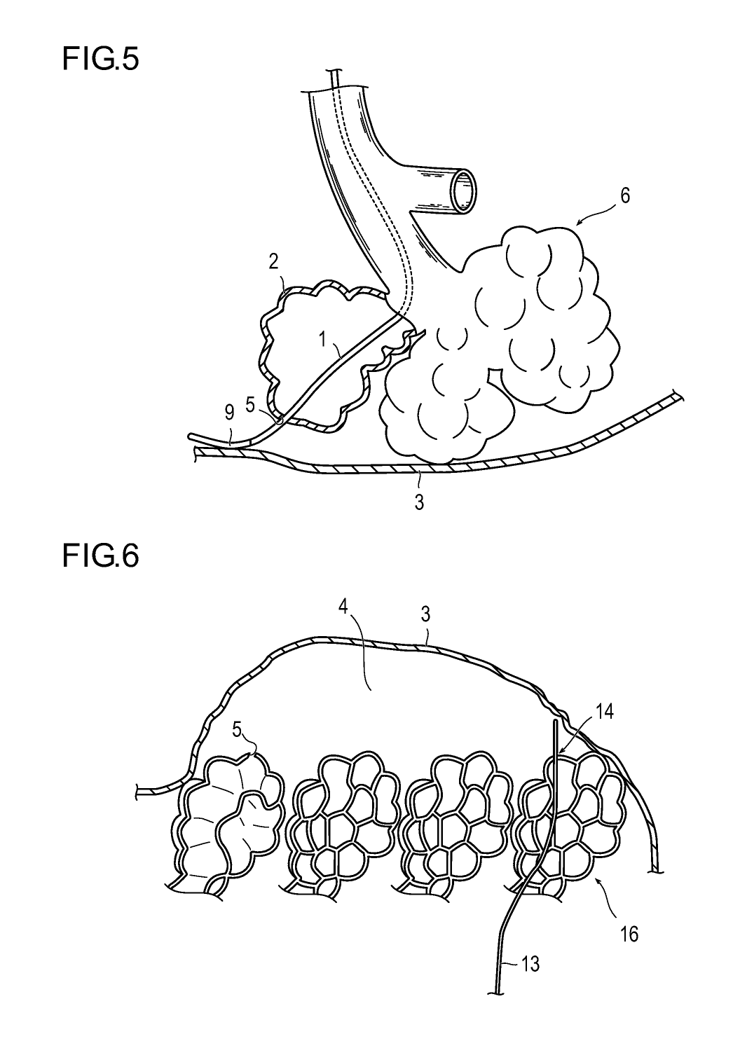

In this step, as shown in FIG. 6, second through-hole forming means 13 is inserted and placed in normal lung parenchyma 16 or normal airway, and a second through-hole 14 is formed in the lung parenchyma 16 or airway by puncture. Specifically, the second though-hole forming means 13 is inserted via the patient's oral cavity or nasal cavity into bronchial tube or bronchiole communicating with a respiratory region contained in normal alveoli or alveolar sacs (hereafter referred also to simply as “normal lung parenchyma”) or normal airway. Then, it is checked, under radioscopy if necessary, whether the distal portion of the second through-hole forming means 13 has reached a desired site. Thereafter, the second through-hole forming means 13 is pushed out distally, whereby the normal lung parenchyma 6 or normal airway (not shown here) is punctured, and the second through-hole forming means 13 penetrates only the normal lung parenchyma 16 or normal airway without breaking the pleura 3, to form the second through-hole 14 (see FIG. 6).

Besides, in the case of forming the second through-hole in this step, the second through-hole is preferably formed by puncturing such that the detachment space 4 formed in the above-mentioned step (a) or the detachment space 4 expanded by step (f) described later and the normal lung parenchyma or normal airway communicate with each other.

This ensures that the normal alveoli and the emphysematous alveoli communicate with each other through the first through-hole, the second through-hole and the detachment space; as a result, the detachment space inside the pleura near the lung surface constitutes a bypass.

Furthermore, in the case where the detachment space formed in the step (a) or the detachment space expanded in the step (f) described later and the normal lung parenchyma or normal airway are not communicating with each other, the following operation may be conducted. When the second through-hole forming means in the present invention is caused to form the second through-hole by an operation on the proximal end, the pushing-out force may be enhanced to bring the distal portion of the second through-hole forming means into contact with the pleura, and, while checking the situation under radioscopy if necessary, the pleura 3 may be detached from the alveoli to form the detachment space, in the same manner as in the step (a). This ensures that the detachment space formed by the second through-hole forming means and the detachment space formed in the step (a) can be easily made to communicate with each other by the step (f) described later or the like operation.

The configuration of the second through-hole forming means is the same as that of the first through-hole forming means used in the step (a); therefore, the description of the configuration is omitted here.

In addition, the filamentous body as a preferred embodiment of the second through-hole forming means in the present invention may also be used as a guide wire in the present invention.

“Step (c)”

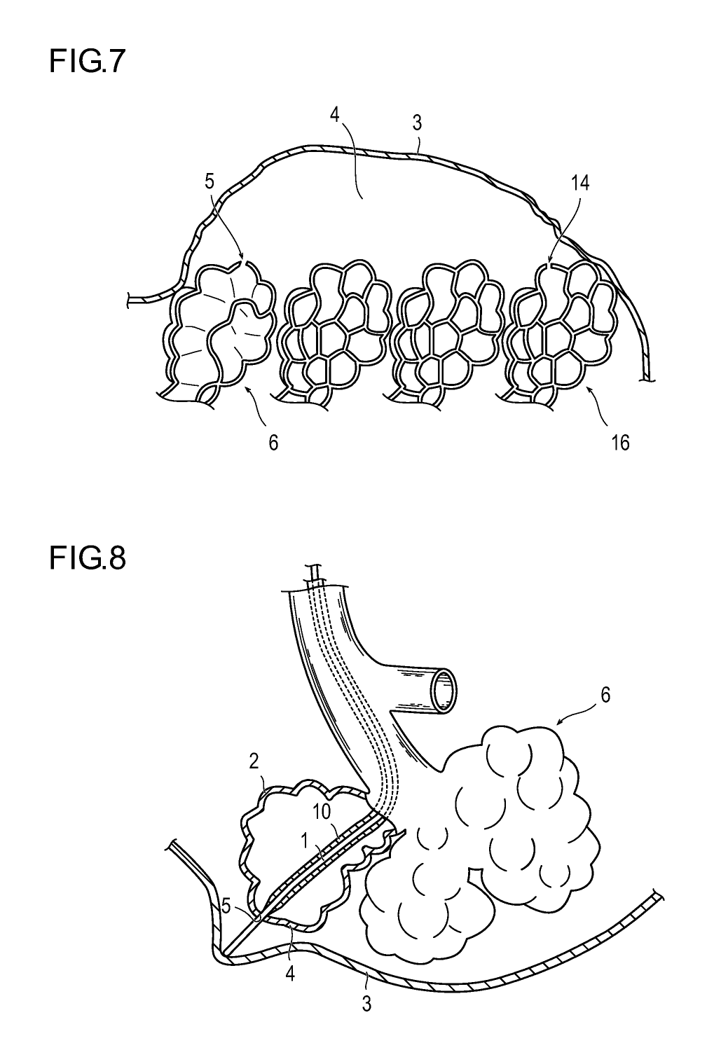

In this step, the first through-hole and the second through-hole are made to communicate with each other via the detachment space formed in the above-mentioned step (a). Specifically, while checking the situation under radioscopy if necessary, a known guide wire or a filamentous body as a preferable embodiment of the first through-hole forming means or the second through-hole forming means is inserted through the patient's oral cavity or nasal cavity into the respiratory region, is passed through the first through-hole 5, is then passed through the detachment space 4, and is passed through the second through-hole 14. In this instance, it is confirmed that the normal lung parenchyma 16 or airway and the emphysematous lung parenchyma 6 or airway are communicating with each other through the detachment space 4. In the case where the normal lung parenchyma 16 or airway (not shown here) and the emphysematous lung parenchyma 6 or airway (not shown here) are communicating with the detachment space 4, the situation is as shown in FIG. 7.

Incidentally, in the method for confirmation as above-mentioned a known guide wire or the above-mentioned filamentous body may be inserted and passed sequentially through the second through-hole, the detachment space and the first through-hole in this order, so as to check whether or not the first and second through-holes are communicating with each other via the detachment space.

After the confirmation, in the case where the detachment space formed by the first through-hole forming means and the normal lung parenchyma or airway are not communicating with each other, the step (f) described later may be carried out wherein detachment space expanding means is inserted through the patient's oral cavity or nasal cavity to be placed in a respiratory region, is then passed through the first through-hole or the second through-hole, and the lung parenchyma and the pleura are detached from each other by the detachment space expanding means, thereby expanding the detachment space.

By the above-mentioned steps (a) to (c), the emphysematous lung parenchyma or airway and the normal lung parenchyma or airway are made to communicate with each other via the detachment space. As a result, the detachment space near the pleura plays the role of a bypass, and air in the emphysematous lung parenchyma or airway flows through the detachment space into the normal lung parenchyma or airway, so that the air can be discharged to the outside of the patient's body.

In addition to the steps (a) to (c) which are components of the present invention, step (d) and/or step (e) described later may be performed optionally. Further, in addition to the steps (k) and (l), step (d) described later may be conducted optionally.

“Step (d)”

In this step (d), after the above-mentioned step (a), the first through-hole is preferably broadened by a first through-hole broadening member for broadening the through-hole, with the first through-hole forming means as a guide. Specifically, in this step, as shown in FIG. 8, with the first through-hole forming means 1 as a guide, the first through-hole 5 is broadened by a first through-hole broadening member 10 for broadening the first through-hole 5. Preferably, with the first through-hole forming means 1 as a guide, the first through-hole 5 is broadened by the first through-hole broadening member 10 which is tapered off on the distal end thereof.

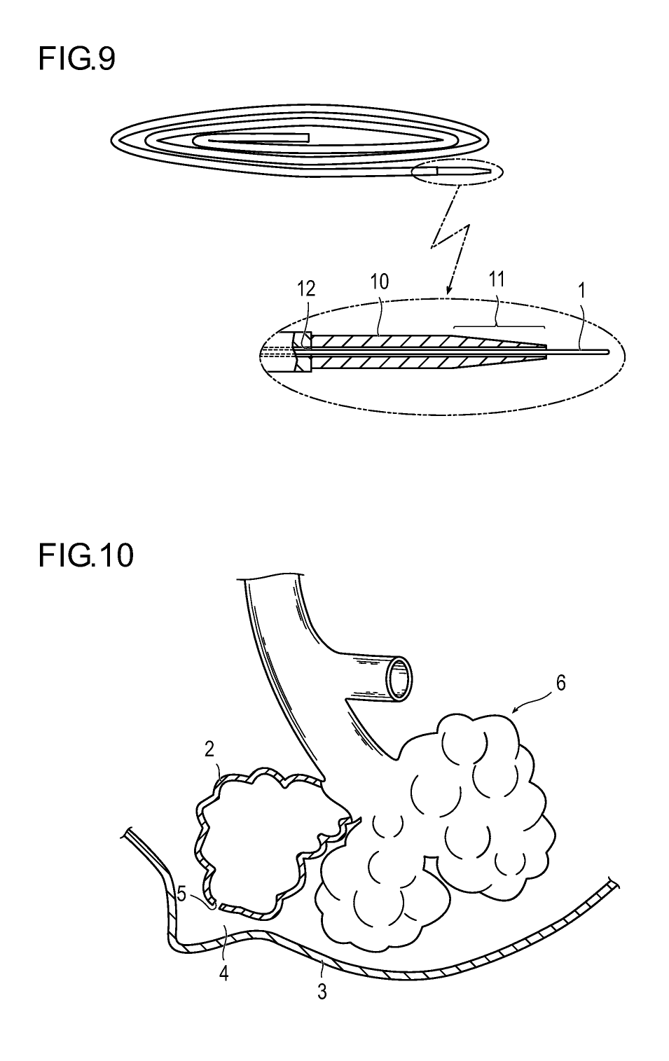

More specifically, for example, as shown in FIG. 9, a tubular first through-hole broadening member 10 is prepared which has a tapered section 11 at the distal end thereof and has openings at both ends thereof. The first through-hole forming means 1 is inserted and passed in the through-hole broadening member 10 so as to be advanceable in the axial direction. The through-hole broadening member 10 is inserted through the patient's oral cavity or nasal cavity into the respiratory region, with the first through-hole forming means 1 as a guide. Alternatively, a first through-hole broadening member wherein a pressure feeder for injecting a filler as known is attached to a pressure feed tube of a double tube structure, with a balloon attached to the distal end thereof, may be inserted through the patient's oral cavity or nasal cavity into the respiratory region. Thereafter, while checking the situation under radioscopy if necessary, the through-hole broadening member 10 is pushed out distally and operated. In this manner, while the first through-hole forming means 1 passed through the through-hole broadening member 10 exhibits a self-aligning function, the tapered section of the through-hole broadening member 10 is fitted into the first through-hole 5, whereby the first through-hole 5 can be broadened (see FIG. 10).

As a result, the through-hole can be formed in the lung parenchyma or airway, and the diameter of the through-hole can be enlarged to a desired size. In addition, when the first through-hole forming means 1 and the first through-hole broadening member 10 are pulled out, it is confirmed that the broadened first through-hole is formed in the alveoli, as for example shown in FIG. 10.

Besides, since the first through-hole broadening member with the balloon attached to the distal end thereof is also of a double tube structure, by inflating the balloon at the distal end in the state of being fitted in the first through-hole with the first through-hole forming means exhibiting a self-aligning function, it is possible to broaden the through-hole.

Therefore, the first through-hole broadening member for use in this step is preferably provided with at least one lumen by which it is guided by the first through-hole forming means and in which the first through-hole forming means is inserted and passed.

As for the structure of the first through-hole broadening member in the present invention, as shown in FIG. 9, the first through-hole broadening member is a tubular body having at least one lumen 12, and is preferably formed with a taper shape 11 (is tapered off) at the distal end thereof. This ensures that the filamentous body 1 composed of a body wire with the flexible section attached to the distal end thereof can be inserted and passed in the lumen 12 so as to be axially movable forward and backward.

The length of the tapered section of the first through-hole broadening member in the present invention is preferably 1 to 50 mm, more preferably 3 to 20 mm.

When the length of the tapered section of the through-hole broadening member is in the range of 1 to 50 mm, in view of the diameter of the broadened first through-hole being not more than about 20 mm, it is considered to be preferable to secure a certain degree of taper length for smooth (low-resistance) broadening of the through-hole.

The overall length of the first through-hole broadening member in the present invention is preferably 200 to 1,000 mm, which is slightly shorter than (or roughly equivalent to) the length of the filamentous body as a preferable embodiment of the first through-hole forming means, from the viewpoint of insertion and passage of the filamentous body in the first through-hole broadening member.

In this case, the first through-hole broadening member is preferably operated in the condition where the first through-hole forming means is preliminarily inserted therein.

In addition, a joint part by which an extension filamentous body similar in length to the above-mentioned filamentous body can be joined may be provided at a proximal portion of the above-mentioned filamentous body. This ensures that even where the first through-hole broadening member is not accompanied by the first through-hole forming means preliminarily inserted therein, the first through-hole broadening member can be advanced to the detachment space 4 with the first through-hole forming member as a guide, in the condition where the distal end position of the first through-hole forming means is fixed at the detachment space 4. This is not restrictive. For instance, the length of the first through-hole forming means may be set to be not less than two times the length of the first through-hole broadening member; this also permits the same operation as mentioned just above.

The outside diameter at the distal end of the taper shape of the through-hole broadening member in the present invention is preferably 0.05 to 2.0 mm, more preferably 0.3 to 0.9 mm, and further preferably 0.4 to 0.7 mm.

The outside diameter at the proximal end of the taper shape of the through-hole broadening member in the present invention is preferably 0.1 to 3.0 mm, more preferably 0.3 to 2.0 mm, and further preferably 0.4 to 0.9 mm.

The material for the first through-hole broadening member in the present invention is preferably the same as that for the flexible section mentioned above. Therefore, setting forth of examples of the material is omitted here.

Another embodiment of the though-hole broadening member in the present invention is one wherein a pressure feeder for injecting a filler is attached to a pressure feed tube of a double tube structure, with a balloon attached to the distal end thereof. The inside of the pressure feed tube and the inside of the balloon are communicating with each other. Furthermore, as the pressure feed tube, one accompanied by a pressure feeder such as a cylinder or pump connected thereto may also be used. In addition, such a structure may be the same as a known balloon catheter structure. In this case, the diameter and the length of the balloon before expansion are the same as those of the tapered-off structure.

“Step (e)”

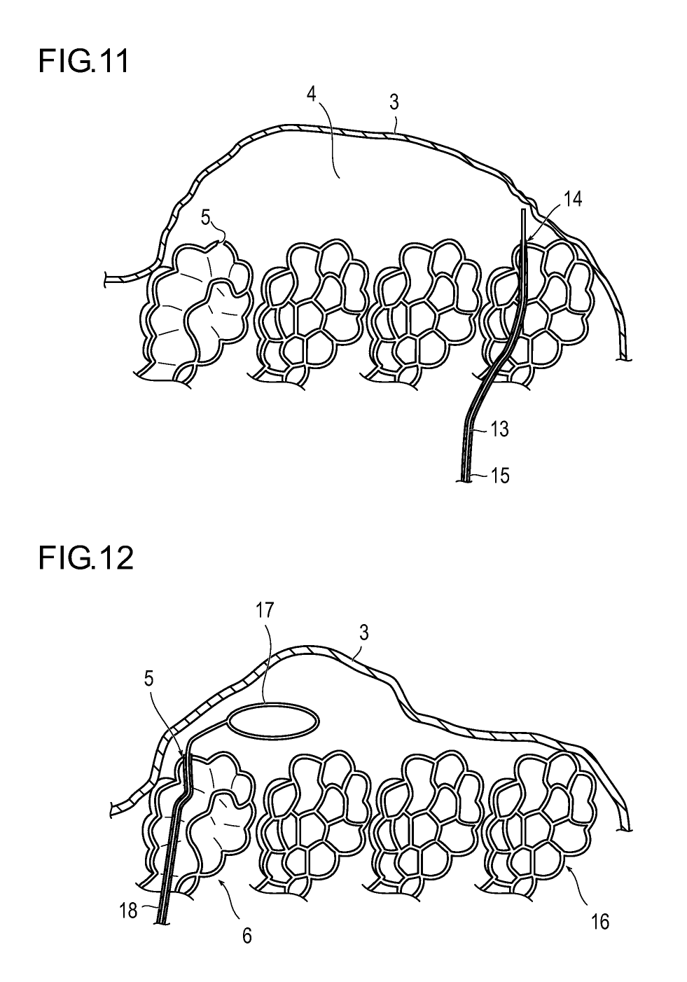

In this step (e), after the above-mentioned step (b), the second through-hole is preferably broadened by a second through-hole broadening member for broadening the through-hole, with the second through-hole forming means as a guide. Specifically, in this step, as shown in FIG. 11, the second through-hole 14 is broadened by a second through-hole broadening member 15 for broadening the second through-hole 14, with the second through-hole forming means 13 as a guide. Preferably, the second through-hole 14 is broadened by a second through-hole broadening member tapered off at the distal end thereof, with the second through-hole forming means as a guide. Specifically, a tubular body having a tapered section at the distal end thereof and having openings at both ends thereof is prepared as a second through-hole broadening member, and the second through-hole forming means is inserted and passed in the through-hole broadening member so as to be axially movable forward and backward. In this manner, the second through-hole broadening is inserted through the patient's oral cavity or nasal cavity into the respiratory region, with the second through-hole forming means as a guide. Thereafter, while checking the situation under radioscopy if necessary, the second through-hole broadening member is pushed out distally and operated. In this manner, while the first through-hole forming means inserted and passed in the through-hole broadening member exhibits a self-aligning function, the tapered section of the second through-hole broadening member is fitted into the second through-hole, whereby the second through-hole can be broadened.

In addition, like in the step (d), a first through-hole broadening member wherein a pressure feeder for injecting a filler is attached to a pressure feed tube of a double tube structure, with a balloon attached to the distal end thereof, may be inserted through the patient's oral cavity or nasal cavity into the respiratory region.

This makes it possible to form the through-hole in the lung parenchyma or airway and to enlarge the diameter of the through-hole to a desired size.

The second through-hole broadening member is the same as the first through-hole broadening member used in the step (d) above, and, therefore, description thereof is omitted here.

Incidentally, the order in which the step (a) and the step (d) are carried out may be reversed. Specifically, a process may be adopted in which after the position of the emphysematous lung parenchyma or emphysematous airway is checked under radioscopy, the second through-hole forming means in the present invention is delivered to normal lung parenchyma or airway in the vicinity of the emphysematous part, thereby forming the second through-hole first. Next, the second through-hole is broadened by the second through-hole broadening member if necessary, and, further, the pleura is detached from the alveoli, thereby forming the detachment space. Thereafter, the first through-hole forming means is made to reach the target site, or the emphysematous lung parenchyma or emphysematous airway, to form the first through-hole in the emphysematous part, whereby the first through-hole and the second through-hole are made to communicate with each other through the detachment space. In this case, optionally, step (f) described later may be conducted to enlarge the detachment space.

In the treatment method according to the present invention, after the above-mentioned step (b), it is preferable to perform step (f) of inserting a detachment space expanding means for expanding the detachment space into the detachment space through the broadened first through-hole or the broadened second through-hole, and expanding the detachment space. Now, the step (f) will be described below.

“Step (f)”

In this step, after the first (or second) through-hole forming means and the first (or second) through-hole broadening member are pulled out, or in the condition where the first (or second) through-hole broadening member is left in the respiratory region, the detachment space expanding means is inserted through the patient's oral cavity or nasal cavity or the like. Thereafter, if necessary, it is checked under radioscopy whether or not the distal portion of the detachment space expanding means has been delivered through the first through-hole or the second through-hole into a gap between the lung parenchyma and the pleura (or to the inside of the detachment space). Then, the detachment space expanding means is operated to open wide the gap between the lung parenchyma and the pleura (or the detachment space), thereby expanding the detachment space where the pleura has been detached from the lung parenchyma. Incidentally, the detachment space has the role of accumulating air (see FIG. 12).

Incidentally, the stage at which the step (f) is carried out is not specifically restricted insofar as it is after the step (d) or the step (e). Preferably, the step (f) is conducted immediately after the step (d), immediately after the step (e), before the step (c), or in the step (c).

When the detachment space is enlarged by the step (f), it becomes easy to place a bypass (for direct communication between the emphysematous lung part and the normal lung parenchyma or airway) in the detachment space as described later, or to place an indwelling body in the detachment space.

The detachment space expanding means is preferably composed of a pleura detaching element including an expandable and contractible elastic member which is inserted into the detachment space formed by detaching the pleura from the lung parenchyma via the broadened through-hole and expands the detachment space, and an operating member by which the elastic member is operated from the proximal end.

The pleura detaching element is preferably a long operating member having an expandable and contractible elastic member at the distal end thereof, and is preferably of such a structure that it can be contained in a catheter.

The pleura detaching element in the present invention is of a structure wherein an expandable and contractible elastic member is attached to the distal end of a long operating member. The expandable and contractible elastic member is preferably an annular body, a balloon-like expansion body or the like, and is more preferably an annular body formed of an elastic material or a balloon of a so-called balloon catheter, or the like. The shape of the annular body is not specifically restricted, and may be appropriately selected from among a circular annular shape, polygonal annular shapes and the like. In addition, the operating member is not particularly limited so long as it is a long member; the operating member is preferably a linear material such as wire, a Lube capable of injecting a filler therein, or the like.

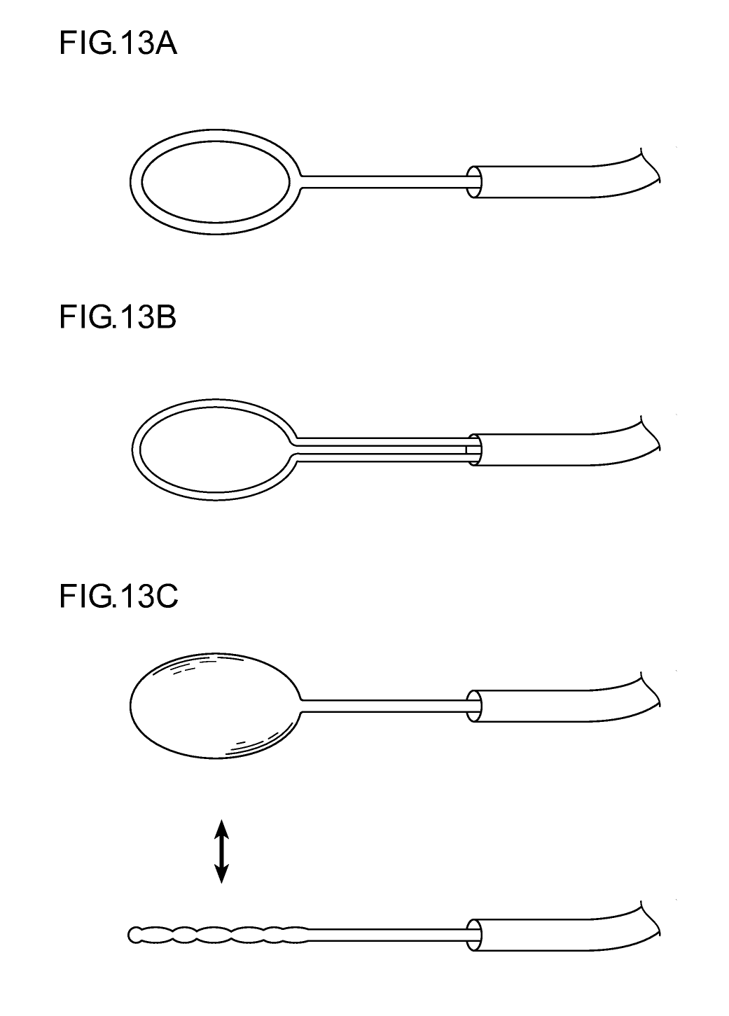

Some preferable embodiments of the pleura detaching element in the present invention are shown in FIGS. 13A to 13C. One preferable embodiment of the pleura detaching element in the present invention is shown in FIG. 13A, wherein a ring-shaped annular body formed of an elastic material is attached to the distal end of a wire serving as an operating member, and this assembly can be contained in a catheter. Thus, when the wire as the operating member is pulled at the proximal end, the ring-shaped annular body is contained into the catheter while being contracted. Further, when the wire is pushed out or the catheter is pulled toward the proximal end, the ring-shaped annular body contained in the contracted state is restored into a normal state.

Another preferable embodiment of the pleura detaching element in the present invention is shown in FIG. 13B, wherein a single linear material formed of an elastic material is formed by bending so as to form a curved portion at the distal end thereof. Thus, an operating member is composed of two wires, which may be twisted or bundled. Like in the embodiment shown in FIG. 13A, the pleura detaching element can be contained into a catheter in a contracted state, and is restored into a normal state when protruded from the catheter.

A further preferable embodiment of the pleura detaching element in the present invention is shown in FIG. 13C, wherein a pressure feeder for injecting a filler is connected to a pressure feed tube accompanied by a balloon attached to the distal end thereof. The inside of the pressure feed tube and the inside of the balloon communicate with each other. In this structure, further, the pressure feeder such as a syringe, an indeflator or a pump is connected to the pressure feed tube. When a filler is added to the pressure feeder such as a syringe, an indeflator or a pump and the pressure feed tube and the pressure feeder is operated after the pressure feeder or the pressure feed tube is protruded to bring the balloon out of the catheter, the filler is injected through the pressure feed tube into the balloon, whereby the balloon can be inflated. Further, after the inflation of the balloon, the balloon can be contracted by sucking out the filler therefrom.

As for the size of the annular body, the maximum outside diameter when the annular body is expanded is preferably 0.5 to 100 mm, more preferably 3 to 50 mm.

The material for the annular body and the operating member is the same as that for the body wire mentioned above, and, therefore, setting forth of examples of the material is omitted here.

The length of the operating member in the present invention is preferably 200 to 1,400 mm, more preferably 200 to 1,000 mm.

In addition, where the operating member is a pressure feed tube, its inside diameter is preferably 0.2 to 3.0 mm, more preferably 0.4 to 1.5 mm, and its outside diameter is preferably 0.5 to 4.0 mm, more preferably 0.7 to 2.5 mm.

As for the size of the balloon, the radial length of the balloon when deflated is preferably 0.5 to 4.0 mm, and the radial length when inflated is preferably 0.7 to 3.0 mm.

When the above-mentioned pleura detaching element is inserted into the through-hole formed to penetrate the lung parenchyma or airway and the elastic member at its distal end is protruded from the catheter to be located between the pleura and the lung parenchyma, the elastic member at the distal end can be expanded. This ensures that the detachment space as an area formed by detaching the pleura from the alveoli can be formed according to the expanded size of the elastic member, or that the detachment space can be enlarged (or the area formed by the detachment can be broadened) by operating the pleura detaching element on the proximal end. Further, by pulling the pleura detaching element, the elastic member can be contained into the catheter, and the elastic member can be contracted. Therefore, by operating the operating member on the proximal end of the pleura detaching device, the elastic member at the distal end can be protruded from and retracted into the catheter, and the elastic member can be controlled in a freely expandable and contractible manner.

Incidentally, since the emphysematous lung parenchyma system, airway system and the area formed by the detachment are connected to each other through the through-hole, air flows into the area formed by the detachment, so that the detachment space is considered to be an air accumulation area.

The material for the balloon is preferably, for example, polyvinyl chloride, polyurethane elastomer, nylon, PET, or rubber, silicone or the like.

In addition, the balloon may contain a radiopaque contrast agent or the like; alternatively, a radiopaque contrast agent or the like may be injected into the balloon when the balloon is inflated. This ensures that the degree of inflation of the balloon can be checked under fluoroscopy, so that the detachment space can be formed assuredly and easily. Here, the contrast agent is not specifically restricted insofar as it is opaque to radiant rays; thus, known radiopaque materials can be used. Specifically, the radiopaque materials described in the column of the step (a) above can be used, and, therefore, setting forth of examples of the radiopaque material is omitted here. These radiopaque materials may be used either singly or in the form of a mixture of two or more of them. Alternatively, a contrast agent layer formed by using a contrast agent (such as above-mentioned) as a base material may be provided on the balloon.

The contrast agent is not particularly limited so long as it is safe for the patient; thus, known contrast agent materials in the form of liquid (inclusive of gelled material) or gas or the like can be used.

The catheter for use in the present invention is a tubular long member having openings on the distal portion side and the proximal portion side and having at least one lumen on the distal portion side. Thus, the catheter may of any structure, refers to a concept including the above-mentioned first or second through-hole broadening member, is not specifically restricted, and is appropriately selected according to the diameter (the number of times of bifurcation) of the bronchial tube or bronchioles into which it is to be introduced. Specifically, known catheters for respiratory system, circulatory system, digestive system and the like which are used for medical use can be used. In addition, the number of lumens in the catheter is also not particularly limited, and may be appropriately selected. For instance, microcatheters used for passing a guide wire in a stenosis in a blood vessel lumen in the cardiovascular region (e.g., FINECROSS (registered trademark), made by Terumo Corp.), PTCA catheters (e.g., Ryujin Plus OTW (registered trademark), made by Terumo Corp.), the above-mentioned tubular through-hole broadening members, and the like are used. Here, the above-mentioned catheter can be inserted into a bronchial tube lumen through a working lumen of a bronchoscope. However, the use of a bronchoscope is not indispensable, insofar as the catheter can be placed in an arbitrary part. Besides, the catheter may be placed by way of a sheath disposed on the proximal portion side.

The guide wire for use in the present invention is not particularly restricted. Known guide wires for respiratory system, circulatory system, digestive system and the like which are used for medical use can be used. The outside diameter and the like of the guide wire can be appropriately selected according to the size of the lumen of the catheter used and the like factors. Therefore, the guide wire in the present invention includes the above-mentioned filamentous (linear) body composed of the body wire accompanied by the flexible section attached to the distal end thereof for use in the present invention. In addition to this, also usable are those guide wires used for treatment of cardiac blood vessels, for example, the guide wire (hereinafter referred to as GW) of Runthrough (registered trademark) (made by Terumo Corp.; outside diameter: 0.014 inch), etc.

In addition, a member having radiopaque property is preferably disposed at a distal portion of the (body) wire or guide wire for use in the present invention or at the distal end of the catheter for use in the present invention. By observation under fluoroscopy, the positions of the distal ends of the guide wire and the catheter protruding from the distal end of a bronchoscope can be grasped, and the guide wire and the catheter can be guided into a respiratory region which includes emphysematous alveoli or alveolar sacs and which has preliminarily determined by fluoroscopy or CT imaging. Besides, the distal ends of the (body) wire, the guide wire and the catheter for use in the present invention may have a structure for restraint or prevention of adhesion to the inner wall of the respiratory region including the alveoli and alveolar sacs, such as a meshed structure or a multi-holed structure.

Incidentally, in the case where a catheter and/or a guide wire is used in all the steps in the present invention, not only the above-mentioned catheters and guide wires but also known catheters and guide wires can be used according to the doctor's judgment.

A particularly preferred embodiment of this step (f) will be described, referring to FIG. 12. After the first through-hole forming means and the first through-hole broadening member are pulled out or in the condition where the first through-hole broadening member is left in the respiratory region (not shown), if necessary, a guide wire or a catheter is inserted through the patient's oral cavity or nasal cavity or the like to be placed in the vicinity of the first through-hole (not shown). Then, the pleura detaching element 17 having an expandable and contractible elastic member with an annular body at the distal end thereof and a wire connected to the elastic member is inserted through the patient's oral cavity or nasal cavity or the like, in such a manner that it is inserted and passed in the catheter. Thereafter, if necessary, it is checked under radioscopy whether or not a distal portion of the pleura detaching element 17 has reached a gap 4 between the lung parenchyma and the pleura (or the inside of the detachment space 4) by way of the first through-hole 5. Subsequently, by operating the elastic member by use of the wire on the proximal end, the gap 4 between the lung parenchyma and the pleura (or the detachment space 4) is broadened, whereby the detachment space 4 formed by detaching the pleura 3 from the lung parenchyma is expanded (see FIG. 12). Incidentally, the detachment space 4 has the role of accumulating air.

When the detachment space is enlarged by the step (f), it becomes easy to place a bypass (for direct communication between the emphysematous lung part and the normal lung parenchyma or airway) in the detachment space as described later, or to place an indwelling body in the detachment space.

After the steps (a) to (c) according to the present technique, optionally with the steps (d) to (f), are applied to the patient, the first or second through-hole and the detachment between the pleura and the alveoli can be cured, optionally with predetermined medication or the like, in about two or three weeks. Therefore, for some reason such as the patient's condition or prolongation of the period for cure, the technique of the following steps (g) to (j) may further be applied to the patient. Now, the steps (g) to (j) will be described below.

“Step (g)”

In this step, a step (g) of placing a first tubular indwelling body, for maintaining the open state of the first through-hole, in the first through-hole is preferably added, after the broadening of the first through-hole in the step (d).

This makes it possible to restrain or prevent the first through-hole from being obstructed as the emphysematous lung parenchyma or airway is cured with the lapse of time.

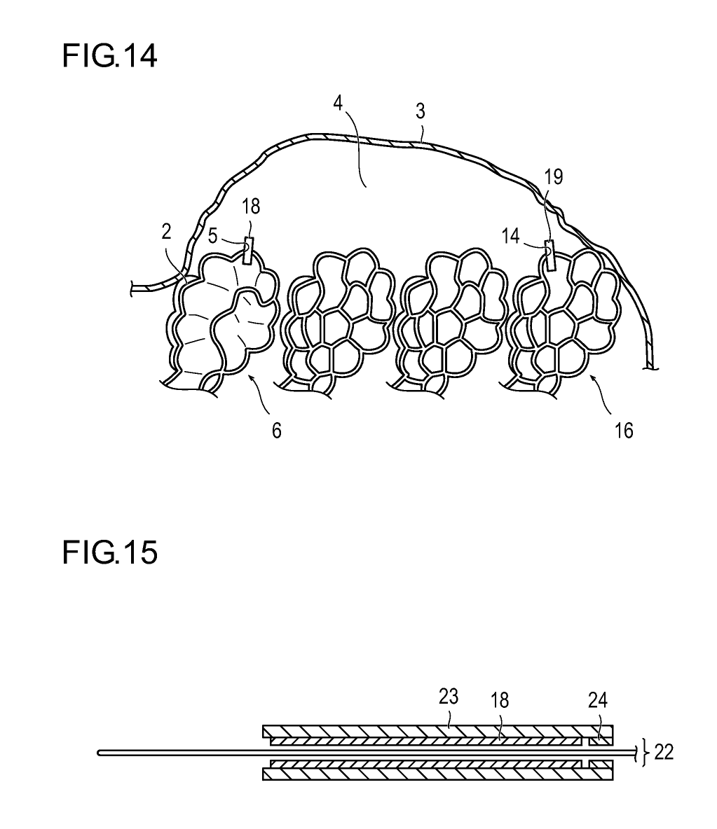

In a preferred embodiment of the step (g), the first tubular indwelling body is placed in the first through-hole by use of a pleural bypass forming element which includes a catheter, a guide wire (inclusive of the first through-hole forming means, here and hereafter) inserted and passed in the catheter, the first tubular indwelling body contained on the distal end in the catheter, and placement means for placing the tubular indwelling body in the through-hole. As a result, as shown in FIG. 14, the first tubular indwelling body 18 is left indwelling in the first through-hole 5.

More specifically, the distal end of the guide wire is inserted and placed through the patient's oral cavity or nasal cavity so as to be located in the first through-hole broadened by the step (d), after which the catheter with the tubular indwelling body contained on the distal end therein is inserted and passed along the guide wire. Then, the distal end of the catheter with the tubular indwelling body contained therein is passed through the first through-hole to be protruded to the detachment space side, by an operation on the proximal end. In addition, the first tubular indwelling body is placed so as to fit in the first through-hole, for example by the placement means described later (a pusher, annular projections provided at predetermined intervals on the tubular indwelling body, weakening lines provided at predetermined intervals on the tubular indwelling body, and a contact member provided on the catheter so as to be engaged with the annular projection). These operations may be conducted under fluoroscopy, if necessary.

This makes it possible to restrain or prevent the through-hole formed in the lung parenchyma or airway from being obstructed as curing of the through-hole progresses with the lapse of time.

It is preferable that the guide wire is inserted and passed in the tubular indwelling body and the catheter.

The placement means in the present invention is preferably composed of a pusher by which the tubular indwelling body contained on the distal end in the catheter is protruded by an operation on the proximal end of the catheter.

The configuration of a preferred embodiment of the placement means in the pleural bypass forming element in the present invention will be described below, referring to the drawings.

As shown in FIG. 15, one embodiment of the pleural bypass forming element in the present invention is as follows. On the distal end in a catheter 23 having at least one lumen 22 located on the outermost side, a first tubular indwelling body 18 is contained so as to make close contact with the inside of the specified lumen 22 in the catheter 23 (inclusive of the case where it is provided with a sheath). Further, a tubular pusher 24 as placement means for placing the tubular indwelling body 18 is disposed on the proximal end of the tubular indwelling body 18. In addition, a guide wire 21 is so disposed as to be inserted and passed in the tubular indwelling body 18 and the tubular pusher 24. Therefore, when the catheter 23 is pulled or the pusher 24 is pushed out after the catheter 23 with the tubular indwelling body 18 in close contact with the inside thereof is inserted into the respiratory region by use of the guide wire 21 as a guide, in the condition where the guide wire 21 is located at a desired position, for example, in the condition where the distal end of the guide wire 21 is passed through the through-hole 5 and located within the detachment space 4, it is possible for the tubular indwelling body 18 to be neatly fitted in the first through-hole 5.

Besides, in such an embodiment, when a plurality of the first tubular indwelling bodies are contained in series with each other in the catheter so as to make close contact with the inside of the catheter, the plurality of indwelling bodies can be placed by a one-time operation. This ensures that not only the tubular indwelling body can be fitted in the first through-hole but also the tubular indwelling body or bodies can be placed in the detachment space, by only controlling the position of the distal end of the guide wire.

In the above-mentioned step (g), the first tubular indwelling body is not specifically restricted insofar as it is a hollow cylindrical body opening at both ends thereof. The length, inside diameter, and outside diameter of the tubular indwelling body are appropriately selected according to the site for placement and the method for placement. For instance, in the case where the tubular indwelling body is placed in the first through-hole by use of pusher type placement means, the tubular indwelling body preferably has an axial length of about 0.4 to 2.0 mm, an outside diameter of about 0.3 to 1.9 mm, and an inside diameter of about 0.2 to 1.8 mm. When the tubular indwelling body is sized within such ranges, it has an inside diameter as large as possible, for promising easy flow of air therethrough, and has such a size that the through-hole can be held thereby.

In addition, the tubular indwelling body is preferably formed from a polymer material, more preferably a biocompatible material or a biodegradable polymer, and further preferably a biodegradable polymer. Examples of the biodegradable polymer include known materials such as aliphatic polyesters such as polylactic acid, polyglycolic acid, etc., cellulose polymers such as cellulose acetate, cellulose nitrate, etc., collagen, polyglycolic acid, polycaprolactone, polyhydroxybutyric acid, polydioxanone, Plastarch material, zein, polydioxane, polylactic acid-glycol copolymer, methylcellulose, and polysaccharides.

Besides, the tubular indwelling body may contain a radiopaque contrast agent or the like. This ensures that the position of the tubular indwelling body can be confirmed under fluoroscopy, so that the tubular indwelling body can be placed in the detachment space, the first through-hole or the second through-hole assuredly and easily.

Besides, the tubular indwelling body may be a one-way valve that the inspirations can inflow only in the detachment space. As a result, it can prevent the inspiration which flowed in the detachment space once from flowing back in the emphysematous lung parenchyma. Incidentally, the method for producing the tubular indwelling body is not specifically restricted, and may be appropriately selected from among the ordinarily used production methods, according to the structure and material of the tubular indwelling body. For example, the method can be selected from among production methods based on the use of etching technique such as laser etching, chemical etching, etc. or laser beam cutting technique. In this case, the annular projections can be formed by known methods such as surface processing, surface treatments, adhesives, etc.

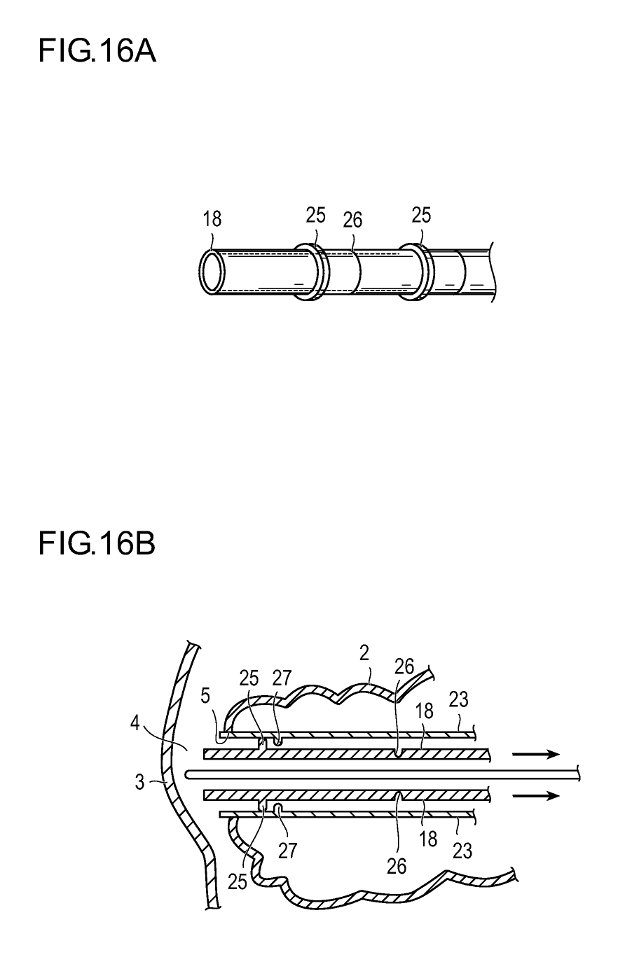

Further, another embodiment of the pleural bypass forming element in the present invention will be described referring to FIGS. 16A and 16B. The first tubular indwelling body 18 is formed at its outer circumferential surface with the annular projections 25 at predetermined intervals, and with the weakening lines 26 (notches) at predetermined intervals. On the other hand, the catheter 24 is provided at its inner circumferential surface with the contact member 27 for engagement with the annular projection 25. Therefore, one of the preferred embodiments of the pleural bypass forming element in the present invention resides in a structure wherein the catheter 23 provided with the contact member 27 at the inner circumferential surface thereof is disposed on the outer side, the tubular indwelling body 18 provided with the annular projections 25 at the outer circumferential surface thereof is fitted inside the catheter 23, and, further, the guide wire 21 is inserted and passed in the tubular indwelling body. Consequently, when the contained tubular indwelling body 18 is pulled after the pleural bypass forming element is inserted by use of the guide wire 21 as a guide, in the condition where the guide wire 21 is disposed at a desired position, for example, in the case where the distal end of the guide wire 21 is passed through the first through-hole 5 and protruded into the detachment space 4, the annular projection 25 and the contact member 27 engage with each other and the weakening member 26 is broken, whereby the tubular indwelling body 18 with a predetermined length can be placed in the through-hole 5 or the detachment space 4.

Incidentally, in such an embodiment, the tubular indwelling body 18 can be formed with the annular projections 25 and the weakening lines 26 at predetermined intervals, so that a plurality of indwelling bodies can be placed by a one-time operation. Furthermore, the placement means for placing the tubular indwelling body in this embodiment in the first through-hole is composed of the annular projections 25 provided at predetermined intervals, the weakening lines 26 provided at predetermined intervals, and the contact member for engagement with the annular projection.

In addition, the catheter in FIG. 16B can be formed with the contact member by a known method, such as application of surface processing or an adhesive or the like to the inside of the catheter.

In the case where the tubular indwelling body is placed in the first through-hole by the placement means based on the use of the weakening line in FIGS. 16A and 16B, the tubular indwelling body is provided with the weakening lines and the annular projections at intervals of 0.2 to 1.9 mm. The tubular indwelling body preferably has an overall axial length of about 200 to 1,000 mm, an outside diameter of about 0.3 to 1.9 mm, and an inside diameter of about 0.2 to 1.8 mm. Besides, in the case where one indwelling body is placed by a one-time operation, it suffices that one weakening line is present at a position 0.2 to 1.9 mm distant from the distal end. When the tubular indwelling body is sized within such ranges, it has an inside diameter as large as possible, for promising easy flow of air therethrough, and has such a size that the through-hole can be held thereby.

Another embodiment of the placement means of the pleural bypass forming element in the present invention may be as disclosed in JP-T-2008-510594. In this case, such means may be used, the means such that an implant and a pusher in an implant delivery system equipped with a heater are joined to each other by a tether or the like so that the joint part can be detached by giving thermal energy to the heater disposed near the joint part, as required.

In addition, the pleural bypass forming element and the placement means thereof in the present invention ensure that not only the tubular indwelling body can be fitted in the through-hole but also the tubular indwelling body or bodies can be placed in the detachment space, by simply controlling the position of the distal end of the guide wire.

“Step (h)”

In the method for treatment of emphysema according to the present invention, it is preferable to add step (h) of placing a second tubular indwelling body 19, for maintaining the open state of the second through-hole 14, in the second through-hole 14, after the broadening of the second through-hole 14 by the step (e) (see FIG. 14).

This makes it possible to restrain or prevent the second through-hole from being obstructed as the emphysematous lung parenchyma or airway is cured with the lapse of time.

Besides, the second tubular indwelling body 18 may be a one-way valve that the inspirations can inflow only in the normal alveoli from the detachment space. This ensures that it can prevent the inspiration from flowing in the detachment space from the normal alveoli.

In a preferred embodiment of the step (h), like in the step (g), the second tubular indwelling body is placed in the second through-hole by use of the pleural bypass forming element which includes the catheter, the guide wire (inclusive of the second through-hole forming means, here and hereafter) inserted and passed in the catheter, the second tubular indwelling body contained on the distal end in the catheter, and the placement means for placing the tubular indwelling body in the through-hole. More specifically, an operation is conducted such that the distal end of the guide wire is located in the second through-hole broadened by the step (e), after which the catheter with the second tubular indwelling body contained on the distal end therein is inserted and passed along the guide wire. Then, the second tubular indwelling body is placed so as to fit in the second through-hole (see FIG. 14), for example by the placement means described above (the pusher, the annular projections provided at predetermined intervals on the tubular indwelling body, the weakening lines provided at predetermined intervals on the tubular indwelling body, the contact member provided on the catheter so as to be engaged with the annular projection, etc.). Therefore, the placement means, the catheter, the guide wire, and the second tubular indwelling body in this step (h) are the same as the placement means, the catheter, the guide wire, and the first tubular indwelling body in the step (g), and, therefore, descriptions of these members are omitted here.

“Step (i)”

In the method for treatment of emphysema according to the present invention, it is preferable to perform step (i) of placing a tubular indwelling body, as a space holding member for holding an expanded state of the detachment space, in the detachment space, after the broadening of the first through-hole or the second through-hole by the step (d) or the step (e).

This makes it possible to restrain or prevent the detachment space from being obstructed as curing progresses with the lapse of time.

In a preferred embodiment of this step (i), a pleural bypass forming element similar to that in the steps (g) and (h) is used. In this case, the tubular indwelling body as the space holding member is placed in the detachment space by use of the pleural bypass forming means which includes the catheter, the guide wire (inclusive of the first through-hole forming means and the second through-hole forming means) inserted and passed in the catheter, the space holding member as the tubular indwelling body contained on the distal end in the catheter, and the placement means for placing the tubular indwelling body in the through-hole.

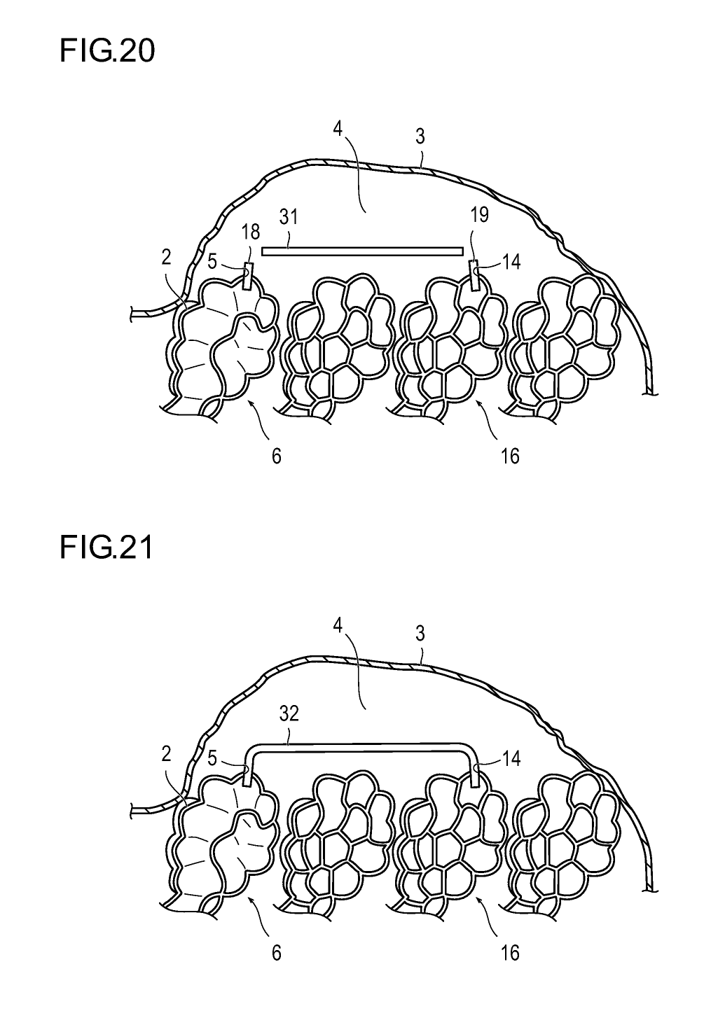

More specifically, after the first through-hole 5 or the second through-hole 14 is broadened by the step (d) or the step (e), the guide wire is inserted through the patient's oral cavity or nasal cavity to be placed so as to pass through the first through-hole 5 or the second through-hole 14. Thereafter, the catheter with the tubular indwelling body as the space holding member 31 contained on the distal end therein is inserted and passed along the guide wire. Then, a position adjustment is conducted so that the space holding member 31 is entirely contained in the detachment space 4 broadened by the step (a) or the step (f). Subsequently, the tubular indwelling body as the space holding member 31 is placed in the detachment space, for example by the above-mentioned placement means (the pusher, the annular projections provided at predetermined intervals on the tubular indwelling body, the weakening lines provided at predetermined intervals on the tubular indwelling body, the contact member provided on the catheter so as to engage with the annular projection, etc.). In this case, the tubular indwelling body also has the role as the space holding member for holding the expanded state of the detachment space. In addition, these operations may be conducted under fluoroscopy, as required. Therefore, the placement means for placing the tubular indwelling body in the through-hole and the placement means for placing the tubular indwelling body in the detachment space may be identical to each other (see FIG. 20).

Thus, the placement means, the catheter, and the guide wire in this step (i) are the same as the placement means, the catheter, and the guide wire in the step (g), and, therefore, descriptions of these members are omitted here. In addition, the tubular indwelling body as the space holding member in the step (i) is the same as the first tubular indwelling body in the step (g), except for size. For example, in the case where the tubular indwelling body as the space holding member is placed in the detachment space by use of the pusher type placement means, the tubular indwelling body preferably have an axial length of about 0.5 to 5.0 mm, an outside diameter of 0.3 to 1.9 mm, and an inside diameter of about 0.2 to 1.8 mm. In the case where the placement is conducted by use of the weakening line type placement means, it is preferable that the weakening lines and the annular projections are provided at intervals of 0.2 to 4.0 mm and that the tubular indwelling body has an overall axial length of about 200 to 1,000 mm, an outside diameter of about 0.3 to 1.9 mm, and an inside diameter of about 0.2 to 1.8 mm. When the tubular indwelling body is sized within such ranges, it has an inside diameter as large as possible, for promising easy flow of air therethrough, and has such a size that the through-hole can be held thereby.

Besides, in the case where one indwelling body is placed by a one-time operation, it suffices that one weakening line is provided at a position 1.0 to 10.0 mm distant from the distal end (a tubular indwelling body length of about 0.4 to 2.0 mm plus a space holding member of about 0.5 to 5.0 mm).

This step (i) ensures that even if the detachment space is obstructed as curing progresses with the lapse of time, air can flow through the inside of the tubular indwelling body placed in the detachment space.

Incidentally, it is preferable to carry out the step (f) before this step (i).

“Step (j)”

In the method for treatment of emphysema according to the present invention, it is preferable to perform step (j) of placing a tubular indwelling body, for forming a communication passage, between the first through-hole and the second through-hole, after the first through-hole and the second through-hole are made to communicate with each other through the detachment space by the above-mentioned step (e).

This ensures that the normal alveoli and the emphysematous alveoli can be made to communicate directly with each other, so that air accumulating in the emphysematous part is exhaled and discharged through the detachment space as the subpleural air accumulation area and the normal alveolar part. In addition, even if the detachment space is obstructed to a certain extent due to curing, the air accumulating in the emphysematous part can be discharged to the outside of the patient's body, since the normal alveoli and the emphysematous alveoli are in direct communication with each other.

In a preferred embodiment of the step (j), the tubular indwelling body is placed so as to establish direct communication between the first through-hole and the second through-hole by use of a pleural bypass forming element similar to that used in the above-mentioned steps (g) to (i).

More specifically, after the above-mentioned step (c), a guide wire (inclusive of the first through-hole forming means and the second through-hole forming means) is inserted through the patient's oral cavity or, nasal cavity and through the emphysematous lung parenchyma or airway to be placed in the first through-hole 5. Further, the guide wire is passed through the detachment space 4 expanded by the step (a) or step (f) and through the second through-hole 14, and the distal end of the guide wire is located in the normal lung parenchyma or airway by an appropriate operation. Then, a catheter with the tubular indwelling body contained on the distal end therein is inserted and passed along the guide wire. In this case, it is confirmed, under fluoroscopy if necessary, that the distal end of the tubular indwelling body extends through the second through-hole 14 to be located in the normal lung parenchyma or airway and that the rear end of the tubular indwelling body extends through the first through-hole 5 to be located in the emphysematous lung parenchyma or airway. Thereafter, the tubular indwelling body is placed in the detachment space (see FIG. 21), for example by use of the above-mentioned placement means (the pusher, the annular projections provided at predetermined intervals on the tubular indwelling body, the weakening lines provided at predetermined intervals on the tubular indwelling body, the contact member provided on the catheter so as to engage with the annular projection, etc.).

Incidentally, in the step (j), the tubular indwelling body may be placed so as to establish direct communication between the first through-hole and the second through-hole, after an operation by which the guide wire is inserted and passed in the second through-hole through the normal lung parenchyma or airway, and further passed through the detachment space formed by the step (c) and through the first through-hole, and by which the distal end of the guide wire is located in the emphysematous lung parenchyma or airway.

Thus, the placement means, the catheter, and the guide wire in this step (j) are the same as the placement means, the catheter, and the guide wire in the step (g), and, therefore, descriptions of these members are omitted here. In addition, the tubular indwelling body as the space holding member in this step (j) is the same as the first tubular indwelling body in the step (g), except for size. For instance, in the case where the placement for establishing the direct communication is conducted by use of the pusher type placement means, the tubular indwelling body preferably has an axial length of about 0.5 to 5.0 mm, an outside diameter of about 0.3 to 1.9 mm, and an inside diameter of about 0.2 to 1.8 mm. When the tubular indwelling body is sized within such ranges, it has an inside diameter as large as possible, for promising easy flow of air therethrough, and has such a size that the through-holes can be held. In the case where the placement for establishing the direct communication is performed by use of the weakening type placement means, it is preferable that the weakening lines and the annular projections are provided at intervals of 0.2 to 4.0 mm, and that the tubular indwelling body has an overall axial length of about 200 to 1,200 mm, an outside diameter of about 0.3 to 1.9 mm, and an inside diameter of about 0.2 to 1.8 mm.

In addition, in the case where one indwelling body is placed by a one-time operation, it suffices that one weakening line is present at a position 1.0 to 10.0 mm distant from the distal end (a tubular indwelling body length of about 0.4 to 2.0 mm plus a space holding member of about 0.5 to 5.0 mm).

This step (j) ensures that even if the detachment space is obstructed as curing progresses with the lapse of time, air can flow through the inside of the tubular indwelling tubular body placed in the detachment space. Incidentally, it is preferable to carry out the step (f) before this step (j).

“Step (k)”

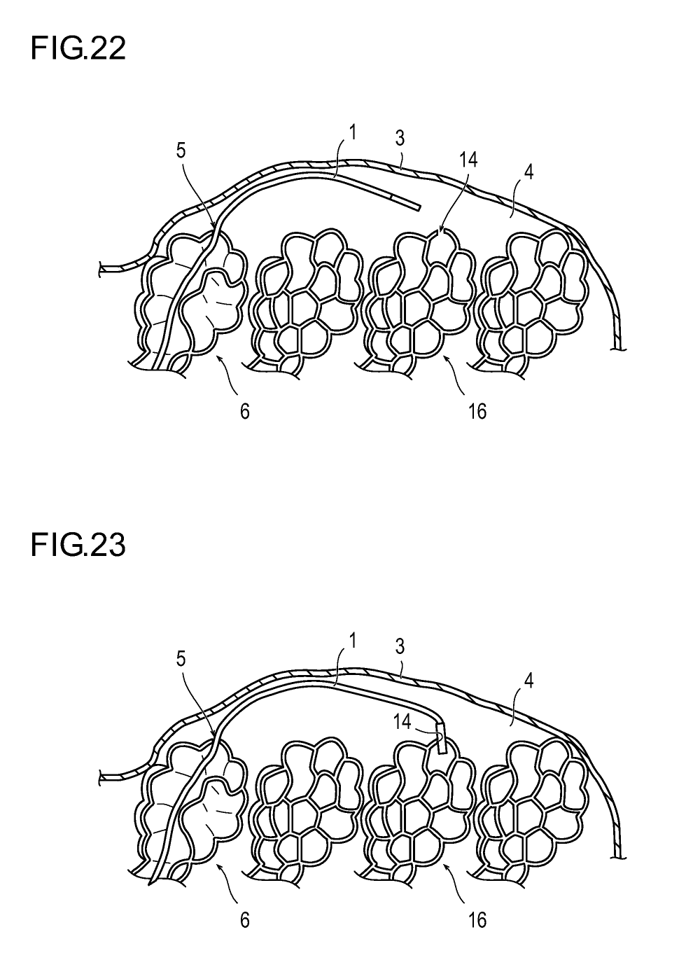

This step is a step of inserting and placing the first through-hole forming means in the normal lung parenchyma or normal airway by way of the detachment space formed by the above-mentioned step (a), and forming the second through-hole in the lung parenchyma or airway by puncture. Specifically, in the step (a), the first through-hole forming means 1 is inserted and placed in the emphysematous lung parenchyma 6 or emphysematous airway, the first through-hole 5 is formed in the emphysematous lung parenchyma 16 or normal airway by puncture, and the first through-hole forming means 1 is pushed so as to put the first through-hole forming means 1 into contact with the pleura 3 and to detach the pleura from the alveoli 2, thereby forming the detachment space 4 (see FIG. 22). Then, the first through-hole forming means 1 is further operated so as to cause its distal portion to extend through the detachment space 4 and puncture the normal lung parenchyma 6 or airway, thereby forming the second through-hole 14 in the normal lung parenchyma or normal airway (see FIG. 23).

This operation by the first through-hole forming means only makes it possible to form the first through-hole in the emphysematous lung parenchyma 6 or emphysematous airway and form the second through-hole in the normal lung parenchyma or normal airway, and to connect the normal lung parenchyma or normal airway and the emphysematous lung parenchyma 6 or emphysematous airway to each other through the detachment space.

In addition, in the case where the step (a) and this step (k) are conducted as the treatment method according to the present invention, the above-mentioned step (d) and/or step (f) may be conducted after the step (a) and before the step (k). Furthermore, in the case where the above-mentioned step (d) and/or step (f) is conducted, the step (g) and/or step (i) may be performed, as required.

On the other hand, after this step (k) is conducted, step (l) and/or the step (h) may be performed in place of the above-mentioned step (e). In this case, the step (l) is a step of inserting the second through-hole forming means other than the first through-hole forming means, inserting and passing the second through-hole forming means in the already formed second through-hole, and then broadening the second through-hole by the second through-hole broadening member while using the second through-hole forming means as a guide. Furthermore, in the case where the step (l) and/or the step (f) is conducted, the step (h) and/or step (j) may be carried out, as required.

In addition, the first through-hole forming means for use in this step is the same as that used in the above-mentioned step (a). Besides, this step also is preferably conducted while checking the situation under fluoroscopy, like the other steps.

A third point of the present invention resides in a pleural bypass forming system for suitable use in the treatment method according to the present invention.

Specifically, the pleural bypass forming system includes:

a lung parenchyma or airway penetrating element including a long body wire capable of being inserted and passed in a respiratory region, and a flexible section which is provided at the distal end of the body wire, penetrates lung parenchyma or airway to form a through-hole, and, upon making contact with pleura, detaches the pleura from the lung parenchyma;

a through-hole broadening element provided at its distal end with a through-hole broadening member for broadening the through-hole; and

a pleura detaching element including an expandable and contractible elastic member which is passed through the broadened through-hole to be inserted into the detachment space formed by detaching the pleura from the lung parenchyma and expands the detachment space, and an operating member for operating the elastic member from the proximal end.

This pleural bypass forming system makes it possible to establish communication between the normal alveoli and the emphysematous alveoli, while using as a bypass the detachment space formed between the lung and the pleura near the lung surface. Consequently, air accumulating in the emphysematous part is exhaled and discharged through the detachment space, which is the subpleural air accumulation area, and the normal alveolar part.

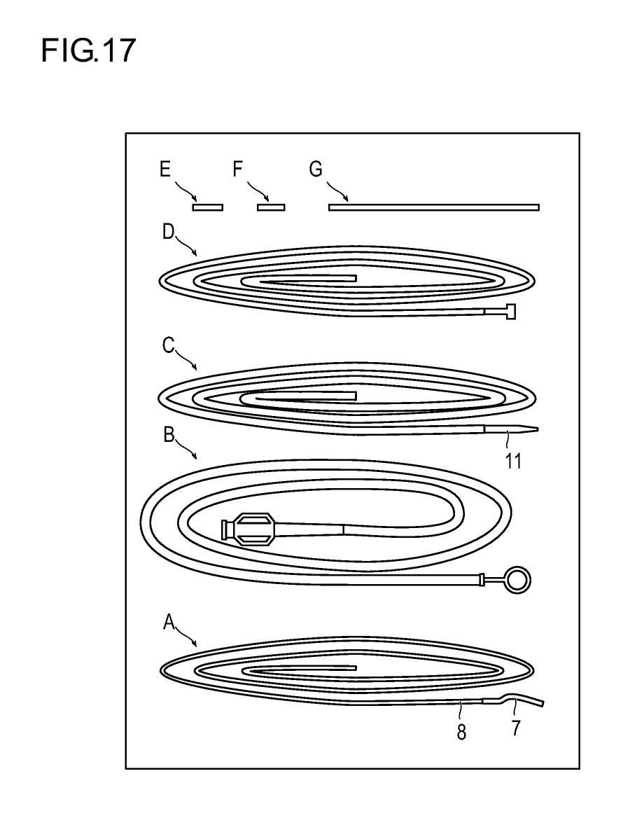

The pleural bypass forming system is shown in FIG. 17. As shown in FIG. 17, the lung parenchyma or airway penetrating element A is suitably used, for example, as the through-hole forming means in the steps (a) and (b). As the structure of the lung parenchyma or airway penetrating element has been described in the step (a) above, the lung parenchyma or airway penetrating element A in the present invention is a filamentous body provided with a flexible section at the distal end thereof. Therefore, the lung parenchyma or airway penetrating element A is not only contained while being inserted in the through-hole broadening element B used in the step (d) and step (e) in such a manner as to be axially movable forward and backward, but also usable as a guide wire. In addition, the flexible section 7 may be so configured that it is provided at the distal end of the body wire 8, penetrates the lung parenchyma or airway, and, upon making contact with the pleura, deforms along the pleura.

Besides, as has been described as a preferred example of the through-hole tip broadening member, the through-hole broadening element B is preferably in the form of a tube with a taper shape 11, and may, if necessary, be contained while being inserted in the catheter 23 so as to be axially movable forward and backward.

Further, the pleura detaching element C, the structure of which has been detailed in the description of a preferred embodiment of the detachment space expanding means in the step (f) above, is contained while being inserted in the catheter 23, in FIG. 17. When the operating member is pulled on the proximal end, the annular body at the distal end of the pleura detaching element C can be contractedly contained into the catheter 23.

In addition, the pleural bypass forming system may further include a pleural bypass forming element which includes a guide wire inserted into the detachment space through the broadened through-hole, a catheter having at least one lumen permitting the guide wire to be inserted and passed in the inside thereof, a tubular indwelling body contained on the distal end in the catheter, and placement means for placing the tubular indwelling body in the through-hole.

In this case, the length and material and the like of the tubular indwelling body contained in a distal end of the catheter are selected according to the purpose of use of the tubular indwelling body. For instance, tubular indwelling bodies E and F in FIG. 17 are for placement in the (first or second) through-hole 5 and 14, whereas a tubular indwelling body G in FIG. 17 is for use to establish communication between lung parenchyma parts or between airway parts, or for use as a space holding member in the detachment space.

Furthermore, as has been described in the step (g) above, the placement means for placing the tubular indwelling body in the through-hole or the detachment space is composed of a pusher D by which the tubular indwelling body contained on the distal end in the catheter is protruded by an operation from the proximal end of the catheter.

Besides, examples of placement means other than the pusher include the tubular indwelling body provided with the weakening line(s), and the technology described in JP-T-2008-510594, as mentioned in the step (g) above.

Now, the embodiment in which emphysematous lung parenchyma or airway and normal lung parenchyma or airway are made to communicate with each other by the pleural bypass forming system according to the present invention will be described below, in the state of being classified into four types, mainly.

### First Embodiment

### Procedure (1):

A radiopaque wire with a flexible distal section, as the lung parenchyma or airway penetrating element in the present invention, is inserted through the patient's oral cavity or nasal cavity and, while checking under radioscopy, it is confirmed that the distal section of the wire with flexible distal section reaches a peripheral emphysematous part. Then, the wire is protruded to directly perforate the alveoli, thereby forming a first through-hole, and, further, it is radibscopically confirmed that the distal section has reached the pleura. In this case, when the distal end of the wire comes into contact with the pleura, part of the pleura is detached from the alveoli or the like, so that a detachment space 4 capable of air accumulation is formed (corresponding to the step (a)). Incidentally, in this procedure (1), the step of forming the first through-hole 5 and the step of detachment of the pleura from the alveoli or the like may be carried out separately, as required.

### Procedure (2):

After the contact of the distal end of the wire with the pleura 3, the wire is inserted and passed in a specified lumen of the through-hole broadening element in the present invention. Then, the through-hole broadening element is slid toward the distal end while using the wire as a guide, to fit the tapered section into the through-hole, thereby broadening the first through-hole (corresponding to the step (d)).

### Procedure (3):

Thereafter, the pleura detaching element in the present invention is inserted and passed in the lumen of the through-hole broadening element from which the wire has been pulled out, or in another lumen of the through-hole broadening element. Then, the operating member on the proximal end is operated so that the expandable and contractible elastic member at the distal end of the pleura detaching element passes through the through-hole. Subsequently, the elastic member is expanded, and the elastic member at the distal end is moved, so as to enlarge the detachment space 4 capable of air accumulation (corresponding to the step (f)).

### Procedure (4):

Subsequently, the above-mentioned wire or another same wire is inserted and passed through the patient's oral cavity or nasal cavity to penetrate normal lung parenchyma or airway, thereby forming a second through-hole 14, and a distal flexible section of the wire is delivered into the detachment space 4 (corresponding to the step (b)). In this case, if the detachment space 4 formed in the procedure (1) is far from the second through-hole, the distal end of the wire is brought into contact with the pleura 3, as required, to detach part of the pleura from the alveoli or the like, thereby connecting the detachment spaces to each other. Alternatively, the procedure (3) is again conducted, or the procedure (3) is conducted after step (5).

### Procedure (5):

Furthermore, with the wire as a guide, the through-hole broadening element used in the procedure (2) or another same through-hole broadening element is slid toward the distal end, to broaden the second through-hole (corresponding to the step (e)).

### Procedure (6):

Finally, it is radioscopically confirmed that communication between the first through-hole and the second through-hole has been established through the detachment space formed in the procedure (3) (corresponding to the step (c)). In this case, if communication between the first through-hole 5 and the second through-hole 14 has not been established through the detachment space 4, the above-mentioned procedures (1) to (6) may be repeated a number of times, as required.

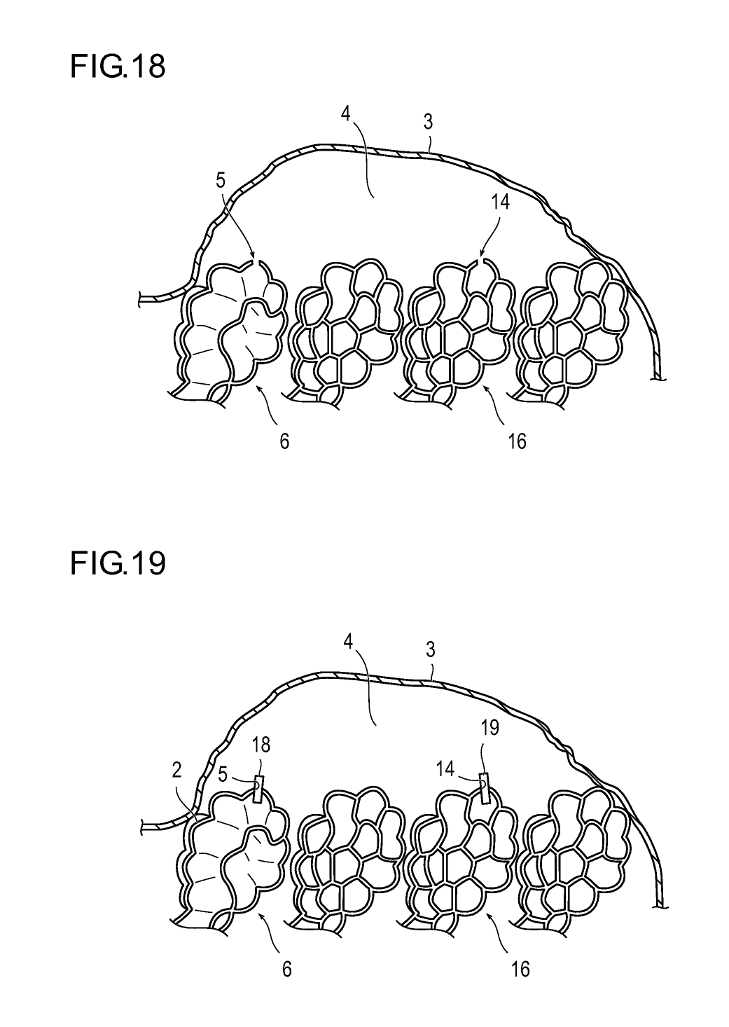

The embodiment where the communication between the emphysematous lung parenchyma or airway and the normal lung parenchyma or airway has been established as a result of the above-mentioned procedures (1) to (6) is as shown in FIG. 18.

### Second Embodiment

This embodiment is the same as the first embodiment, except that step (7) and step (8) are conducted as follows.

### Procedure (7):

After the above-mentioned procedure (2), the pleural bypass forming element in the present invention is so operated as to locate the distal end of a guide wire in the first through-hole formed by the procedure (2). Then, by use of the above-mentioned placement means, a first tubular indwelling body 18 is placed to fit in the first through-hole 5 (corresponding to the step (g)).

### Procedure (8):

After the above-mentioned procedure (4), a second tubular indwelling body 19 is placed to fit in the second through-hole 14 by use of the pleural bypass forming element (corresponding to the step (h)).

In addition, the procedure (7) may be carried out at any time after the step (2). Similarly, the step (8) may be carried out at any time after the procedure (5).

The embodiment where the communication between the emphysematous lung parenchyma or airway and the normal lung parenchyma or airway has been established as a result of the above-mentioned procedures (1) to (8) is as illustrated in FIG. 19.

### Third Embodiment

This embodiment is the same as the second embodiment, except that the procedure (7) and the procedure (8) are performed after procedure (9).

### Procedure (9):

After the above-mentioned procedure (3), the pleural bypass forming element in the present invention is so operated as to locate the distal end of the guide wire in the detachment space formed by the procedure (4), and a tubular indwelling body 31 as a space holding member for holding an expanded state of the detachment space is placed in the detachment space (corresponding to the step (i)).

Therefore, the procedure (7) in this third embodiment may be carried out at any time after the step (9) is conducted. Also, the procedure (8) in this third embodiment may be performed at any time after the procedure (9) and the procedure (4) are conducted.

The embodiment where the communication between the emphysematous lung parenchyma or airway and the normal lung parenchyma or airway has been established as a result of the above-mentioned procedures (1) to (9) is as shown in FIG. 20.

### Fourth Embodiment

This embodiment is the same as the first embodiment, except that procedure (10) is conducted as follows.

### Procedure (10):

After the above-mentioned procedure (6), the guide wire of the pleural bypass forming element in the present invention is inserted and passed in the first through-hole 5 via the emphysematous lung parenchyma or airway, and is further passed through the detachment space 4 formed by the procedure (3) and through the second through-hole 14, and the distal end of the guide wire is located in the normal lung parenchyma or airway, by an appropriate operation. Thereafter, by use of the pleural bypass forming element in the present invention, a tubular indwelling body 32 is so placed as to establish direct communication between the first through-hole 5 and the second through-hole 14 (corresponding to the step (j)).

Incidentally, in the procedure (10), placement of the tubular indwelling body so as to establish direct communication between the first through-hole and the second through-hole may be conducted, after an operation in which the guide wire is inserted and passed in the second through-hole via the normal lung parenchyma or airway, and is further passed through the detachment space formed by the procedure (3) and through the first through-hole, and in which the distal end of the guide wire is located in the emphysematous lung parenchyma or airway.

### Fifth Embodiment

### Procedure (11):

After the procedure (1), the guide wire is further extended in the detachment space 4 formed. Then, while checking under radioscopy, the orientation of the distal end of the wire is adjusted, and the distal end is passed through the detachment space 4 to puncture the normal lung parenchyma or airway, so as to form the second through-hole 14 in the normal lung parenchyma or airway.

After the procedure (1) or after the procedure (11), the procedure (3) and the procedures (6) to (10) may be repeated a number of times, as required.

The embodiment where the emphysematous lung parenchyma or airway and the normal lung parenchyma or airway are made to communicate with each other as a result of the procedures (1) to (6), the procedure (10) and the procedure (11) is as shown in FIG. 21.

### Example

A dog of an emphysema model by an elastase administration method was fixed in supine position on an operating table. A filamentous body with a flexible distal section (for example, RADIFOCUS guide wire, made by Terumo Corp., 0.018 inch) as lung parenchyma or airway through-hole forming means was inserted through the oral cavity. While checking through radioscopy (made by GE), the filamentous body with the flexible distal section was operated and its distal end section was confirmed to reach a peripheral emphysematous part. Then, the filamentous body was protruded to directly puncture the alveoli, thereby forming a first through-hole, and, further, the distal section was radioscopically confirmed to have reached the pleura.

In addition, as above-mentioned, this device has a Runthrough NS distal end abutting load of 1 gf and an outside diameter of 0.014 inch; therefore, puncture is possible when a load of not less than 10 gf/mm² is applied.

After the distal end of the filamentous body is brought into contact with the pleura, the filamentous body was inserted and passed in a specified lumen of a through-hole broadening member, which had a GW outside diameter of 0.46 mm, a catheter inside diameter of 0.50 mm, a catheter outside diameter of 0.55 mm (distalmost end), a catheter outside diameter of 2.00 mm (proximal end), a taper length of 40 mm, and a through-hole diameter of 2.0 mm. With the filamentous body as a guide, the through-hole broadening member was slid toward the distal end so as to fit its tapered section into the through-hole, thereby broadening the through-hole.

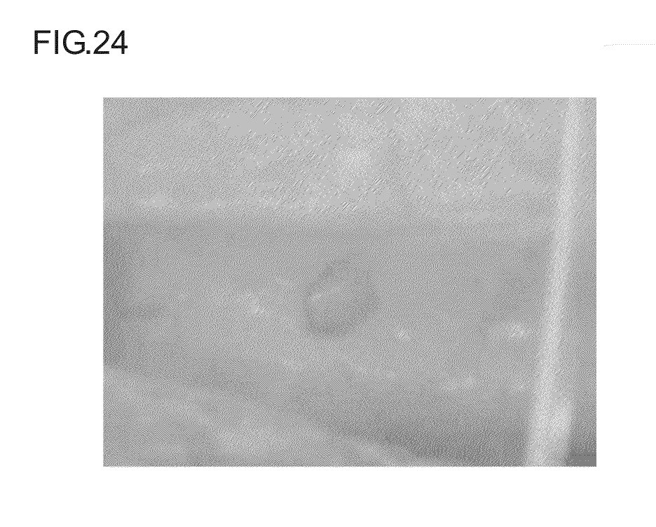

Thereafter, a pleura detaching element was inserted and passed in the lumen of the through-hole broadening member from which the wire was pulled out, or in another lumen of the through-hole broadening member. The pleura detaching element had an annular body attached to the distal end of a wire, wherein the size (diameter) of the annular body when expanded was 5.0 mm, the size (diameter) of the annular body before expansion was not more than 0.5 mm, and the wire (made of NiTi) had a diameter of 0.15 mm. The pleura detaching element was operated so that the elastic member at its distal end is passed through the through-hole, the elastic member was expanded, and the elastic member at the distal end was moved by applying a torque, so as to form a detachment space capable of air accumulation. Thereafter, thoracotomy was performed to expose the lung, and it was checked whether a detachment space had been formed or not, followed by photographing. The result is shown in FIG. 24. It is confirmed from the figure that the pleura has been detached from the lung parenchyma in a central area, thereby forming a detachment space capable of accumulation of air therein.

Subsequently, the region of chest was closed by suture. Then, the wire or another same wire was further inserted and passed through the mouth to penetrate normal lung parenchyma or airway, thereby forming a second through-hole, and delivering its distal flexible section into the inside of the detachment space.

As a result, the normal alveoli and the emphysematous alveoli are made to communicate with each other via the first through-hole, the second through-hole, and the detachment space. Accordingly, communication is established between the normal alveoli and the emphysematous alveoli, by using as a bypass the detachment space formed between the lung and the pleura near the lung surface. This ensures that air accumulating in the emphysematous part is exhaled and discharged via the detachment space, which is a subpleural air accumulation area, and the normal alveolar part.

The present disclosure contains subject matter related to that disclosed in U.S. 61/580,735 filed in the Japan Patent Office on Dec. 28, 2011, the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A method for treatment of emphysema, comprising:
a step (a) of inserting and placing first through-hole forming means in emphysematous lung area, forming a first through-hole in the emphysematous lung area by puncture, and, after contact of the first through-hole forming means with pleura, detaching the pleura from the lung area to form a detachment space;
a step (b) of inserting and placing second through-hole forming means in normal lung area, and forming a second through-hole in the lung area by puncture; and
a step (c) of causing the first through-hole and the second through-hole to communicate with each other through the detachment space formed in the step (a).

2. The method for treatment of emphysema according to claim 1, wherein the lung area is lung parenchyma.

3. The method for treatment of emphysema according to claim 1, wherein the lung area is airway.

4. The method for treatment of emphysema according to claim 1, comprising (d) a step of broadening the first through-hole by a first through-hole broadening member for broadening the through-hole, with the first through-hole forming means as a guide, after the step (a).

5. The method for treatment of emphysema according to claim 1, comprising (e) a step of broadening the second through-hole by a second through-hole broadening member for broadening the through-hole, with the second through-hole forming means as a guide, after the step (b).

6. The method for treatment of emphysema according to claim 1, comprising (f) a step of inserting detachment space expanding means for expanding the detachment space into the detachment space through the first through-hole to expand the detachment space, after the step (a).

7. The method for treatment of emphysema according to claim 1, comprising (f) a step of inserting detachment space expanding means for expanding the detachment space into the detachment space through the second through-hole to expand the detachment space, after the step (b).

8. The method for treatment of emphysema according to claim 1, comprising (g) a step of placing in the first through-hole a first tubular indwelling body for maintaining an open state of the first through-hole, after the first through-hole is expanded in the step (d).

9. The method for treatment of emphysema according to claim 1, comprising (h) a step of placing in the second through-hole a second tubular indwelling body for maintaining an open state of the second through-hole, after the second through-hole is expanded in the step (e).

10. The method for treatment of emphysema according to claim 1, comprising (i) a step of placing in the detachment space a tubular indwelling body as a space holding member for holding an expanded state of the detachment space, after the first through-hole is expanded in the step (d).

11. The method for treatment of emphysema according to claim 1, comprising (i) a step of placing in the detachment space a tubular indwelling body as a space holding member for holding an expanded state of the detachment space, after the second through-hole is expanded in the step (e).

12. The method for treatment of emphysema according to claim 1, comprising (j) a step of placing a tubular indwelling body as a communication tube for forming a communication passage between the first through-hole and the second through-hole, after the step (c) of causing the first through-hole and the second through-hole to communicate with each other through the detachment space.

13. A method for treatment of emphysema, comprising:
a step (k) of inserting and placing first through-hole forming means in emphysematous lung area, forming a first through-hole in the lung area by puncture, and, after contact of the first through-hole forming means with pleura, detaching the pleura from the lung area to form a detachment space; and
a step (l) of inserting and placing the first through-hole forming means in normal lung are by way of the detachment space formed in the step (k), and forming a second through-hole in the lung area by puncture.

14. The method for treatment of emphysema according to claim 13, wherein the lung area is lung parenchyma.

15. The method for treatment of emphysema according to claim 13, wherein the lung area is airway.
